(19) United States
(12) Patent Application Publication
(10) Pub. No.: US 2024/0226213 A1
(43) Pub. Date: Jul. 11, 2024

(54) Treatment of Warts

(71) Applicant: Nielsen BioSciences, Inc., San Diego, CA (US)

(72) Inventor: H. Stewart NIELSEN, JR., San Diego, CA (US)

(73) Assignee: Nielsen BioSciences, Inc., San Diego, CA (US)

(21) Appl. No.: 18/609,229

(22) Filed: Mar. 19, 2024

Related U.S. Application Data
(63) Continuation of application No. 18/304,629, filed on Apr. 21, 2023, now Pat. No. 11,963,991.
Application No. 18/304,629 is a continuation of application No. 17/378,411, filed on Jul. 16, 2021, now Pat. No. 11,666,617.
Application No. 17/378,411 is a division of application No. 16/532,247, filed on Aug. 5, 2019, now Pat. No. 11,116,808.
(60) Provisional application No. 62/714,942, filed on Aug. 6, 2018.
(60) Provisional application No. 62/880,742, filed on Jul. 31, 2019.

(51) Int. Cl. A61K 36/064; A61K 9/00; A61P 17/12

(52) U.S. Cl. CPC A61K 36/064; A61K 9/0019; A61P 17/12

(57) ABSTRACT

Disclosed herein are compositions having standardized potencies for use in the treatment of warts. Methods of treating a common wart comprising administering one or more intralesional injections of compositions having standardized potencies to a patient in need thereof are also disclosed. Further disclosed are methods of treating a non-common wart administering one or more intralesional injections of compositions having standardized potencies to a patient in need thereof.

### CROSS REFERENCE TO RELATED APPLICATIONS

This application claims benefit of U.S. Provisional Application No. 62/714,942, filed Aug. 6, 2018, and U.S. Provisional Application No. 62/880,742, filed Jul. 31, 2019, the entirety of each of which is incorporated by reference herein.

### FIELD OF THE INVENTION

The present disclosure relates to therapeutics having a standardized potency for the complete resolution of common warts and for the prevention of recurrence of common warts.

### BACKGROUND OF THE INVENTION

Common warts are infections of the skin caused by human papillomavirus (HPV). The virus initially targets epidermal basal cells and induces hyperplasia and hyperkeratosis, which presents clinically as a wart. While not life threatening, common warts can cause both physical discomfort and embarrassment in patients.

It is estimated that common warts have an overall prevalence of 2 to 20% in the United States. However, current treatment options for common warts vary in their effectiveness and often result in recurrence. Treatments including topical salicylic acid, topical imiquimod, bleomycin injections, cryotherapy, excision, electrocautery, and laser vaporization are employed with various outcomes and side effects.

Immunotherapy using Candida albicans antigens has also been studied. However, a range of response levels were reported. See Aldahan AS et al., “Efficacy of intralesional immunotherapy for the treatment of warts: A review of the literature,” Dermatologic Therapy 2016; 29:197-207, Table 1; and Alikhan A et al., “Use of Candida antigen injections for the treatment of verruca vulgaris: A two-year mayo clinic experience,” Journal of Dermatological Treatment 2016; 27(4): 355-58, 355, Table 2. The inconsistent results may be due to the use of different sources of Candida antigen in these studies, having apparently different concentrations and potencies. For example, two studies utilized Candida antigens obtained from Hollister-Stier (Alikan et al. at 355 and Perman M et al., “The painful purple digit: an alarming complication of Candida albicans antigen treatment of recalcitrant warts,” Dermatitis: Contact, Atopic, Occupational, Drug 2005; 16(1):38-40, cited in Aldahan et al.), another study utilized Candida antigens obtained from Bayer (Johnson et al., “Intralesional injection of mumps or candida skin test antigens: a novel immunotherapy for warts,” Arch Dematol. 2001; 137:451-55, cited in Aldahan et al. and Alikhan et al.), yet another study utilized Candida antigens obtained from Creative Drug Industries (Majid I and Imran S, “Immunotherapy with Intralesional Candida Albicans Antigen in Resistant or Recurrent Warts: A Study,” Indian Journal of Dermatology 2013: 58(5): 360-65, cited in Aldahan et al. and Alikhan et al.), while other studies utilized Candin® manufactured by Allermed Laboratories (Kim KH et al., “Phase 1 clinical trial of intralesional injection of Candida antigen for the treatment of warts,” Arch Dematol 2010, 146(12): 1431-33; Pfenninger JL and Fowler GC, “Procedures for Primary Care,” Third Edition, Elsevier 2010, 639-43; Signor RJ, “Candida albicans Intralesional Injection Immunotherapy of Warts,” Cutis 2002; 70:185-92, cited in Alikhan et al .; Phillips RC et al., “Treatment of Warts with Candida Antigen Injection,” Arch Dematol 2001, 136(10): 1274-5); Wong A and Crawford RI, “Intralesional Candida antigen for common warts in people with HIV,” J Cutan Med Surg. 2013; 17(5): 313-15, cited in Aldahan et al. Some studies also do not report their sources of Candida antigens. See Summers P et al., “Treatment of recalcitrant verruca vulgaris with Candida antigen in patient with human immunodeficiency virus,” J Drugs Dermatol 2009; 8(3): 268-69, cited in Aldahan et al .; “Warts Refractory to Conventional Therapy Yield to Candida Antigen,” American Academy of Family Practice 90 Clinical Perspectives 1990; Harada S, “Clinical Application of Fungus Extracts and its Culture filtrate in the Treatment of Skin Diseases: (3) Candida Vaccine in the Treatment of Warts,” Japanese Journal of Dermatology 1979; 89(6), 397-402.

In view of the various sources being studied, the dosages reported by volumes and weight by volume (v/w) dilutions therefore do not provide sufficient information to physicians to achieve consistent cure rates of warts. At best, U.S. Pat. No. 6,350,451 to Horn provides dosing of antigens based on initial delayed type hypersensitivity (DTH) responses. See '451 Patent at Example. Therefore, Candida antigen compositions having standardized potencies for treatment of warts are desired.

### SUMMARY OF THE INVENTION

The present disclosure provides for, and includes, compositions having standardized potencies for use in the treatment of one or more common warts or non-common warts. The present disclosure also provides for, and includes, methods for treating one or more common warts or non-common warts.

In an aspect, the present disclosure provides for, and includes a method for treating a common wart in a subject in need thereof, the method comprises administering one or more intralesional injections to the subject of an amount of a pharmaceutical composition effective for complete resolution of the common wart at a cumulative dose of 2.5 units of potency, where the pharmaceutical composition comprises a filtered extract of Candida albicans and secreted antigens.

In an aspect, the present disclosure provides for, and includes a method for treating a common wart in a subject in need thereof, the method comprises administering one or more intralesional injections to the subject of an amount of a pharmaceutical composition capable of complete resolution of the common wart at a cumulative dose of 2.5 units of potency, where the pharmaceutical composition comprises a filtered extract of Candida albicans and secreted antigens.

In an aspect, the present disclosure provides for, and includes a method for treating a common wart in a subject in need thereof, the method comprises administering one or more intralesional injections to the subject of an amount of a pharmaceutical composition effective for partial resolution of the common wart at a cumulative dose of 5 units of potency, where the pharmaceutical composition comprises a filtered extract of Candida albicans and secreted antigens.

In an aspect, the present disclosure provides for, and includes a method for treating a common wart in a subject in need thereof, the method comprises administering one or more intralesional injections to the subject of an amount of a pharmaceutical composition capable of partial resolution of the common wart at a cumulative dose of 5 units of potency, where the pharmaceutical composition comprises a filtered extract of Candida albicans and secreted antigens.

In an aspect, the present disclosure provides for, and includes a method for treating a plurality of common warts in a subject in need thereof, the method comprises administering one or more intralesional injections to the subject of an amount of a pharmaceutical composition effective for partial resolution of the plurality of common warts at a cumulative dose of 5 units of potency, where the pharmaceutical composition comprises a filtered extract of Candida albicans and secreted antigens.

In an aspect, the present disclosure provides for, and includes a method for treating a plurality of common warts in a subject in need thereof, the method comprises administering one or more intralesional injections to the subject of an amount of a pharmaceutical composition capable of partial resolution of the plurality of common warts at a cumulative dose of 5 units of potency, where the pharmaceutical composition comprises a filtered extract of Candida albicans and secreted antigens.

In an aspect, the present disclosure provides for, and includes a method for treating a plurality of common warts in a subject in need thereof, the method comprises administering one or more intralesional injections to the subject of an amount of a pharmaceutical composition effective for reducing the diameter of each of the plurality of common warts by at least 50% at a cumulative dose of 1 unit of potency, where the pharmaceutical composition comprises a filtered extract of Candida albicans and secreted antigens.

In an aspect, the present disclosure provides for, and includes a method for treating a plurality of common warts in a subject in need thereof, the method comprises administering one or more intralesional injections to the subject of an amount of a pharmaceutical composition capable of reducing the diameter of each of the plurality of common warts by at least 50% at a cumulative dose of 1 unit of potency, where the pharmaceutical composition comprises a filtered extract of Candida albicans and secreted antigens.

In an aspect, the present disclosure provides for, and includes a method for treating a non-common wart in a subject in need thereof, where the subject has one or more common warts, the method comprises administering one or more intralesional injections to the subject of an amount of a pharmaceutical composition effective for complete resolution of the non-common wart at a cumulative dose of 5 units of potency, where the pharmaceutical composition comprises a filtered extract of Candida albicans and secreted antigens.

In an aspect, the present disclosure provides for, and includes a method for treating a non-common wart in a subject in need thereof, where the subject has one or more common warts, the method comprises administering one or more intralesional injections to the subject of an amount of a pharmaceutical composition capable of complete resolution of the non-common wart at a cumulative dose of 5 units of potency, where the pharmaceutical composition comprises a filtered extract of Candida albicans and secreted antigens.

In an aspect, the present disclosure provides for, and includes a method for treating a previously treated common wart in a subject in need thereof, the method comprises administering one or more intralesional injections to the subject of an amount of a pharmaceutical composition effective for complete resolution of the previously treated common wart at a cumulative dose of 5 units of potency, where the pharmaceutical composition comprises a filtered extract of Candida albicans and secreted antigens.

In an aspect, the present disclosure provides for, and includes a method for treating a previously treated common wart in a subject in need thereof, the method comprises administering one or more intralesional injections to the subject of an amount of a pharmaceutical composition capable of complete resolution of the previously treated common wart at a cumulative dose of 5 units of potency, where the pharmaceutical composition comprises a filtered extract of Candida albicans and secreted antigens.

In an aspect, the present disclosure provides for, and includes a method for delaying recurrence of a common wart in a subject in need thereof, the method comprises administering one or more intralesional injections to the subject of an amount of a pharmaceutical composition effective for delaying the reappearance of the common wart upon resolution at a cumulative dose of 2.5 units of potency, where the pharmaceutical composition comprises a filtered extract of Candida albicans and secreted antigens.

In an aspect, the present disclosure provides for, and includes a method for delaying recurrence of a common wart in a subject in need thereof, the method comprises administering one or more intralesional injections to the subject of an amount of a pharmaceutical composition capable of delaying the reappearance of the common wart upon resolution at a cumulative dose of 2.5 units of potency, where the pharmaceutical composition comprises a filtered extract of Candida albicans and secreted antigens.

In an aspect, the present disclosure provides for, and includes a medicament comprising a filtered extract of Candida albicans and secreted antigens formulated for complete resolution of a common wart at a cumulative dose of 2.5 units of potency.

In an aspect, the present disclosure provides for, and includes a medicament comprising a filtered extract of Candida albicans and secreted antigens formulated for partial resolution of a common wart at a cumulative dose of 5 units of potency.

In an aspect, the present disclosure provides for, and includes a medicament comprising a filtered extract of Candida albicans and secreted antigens formulated for reducing the diameter of a common wart by at least 50% at a cumulative dose of 1 unit of potency.

In an aspect, the present disclosure provides for, and includes a medicament comprising a filtered extract of Candida albicans and secreted antigens formulated for partial resolution of a plurality of common warts at a cumulative dose of 5 units of potency.

In an aspect, the present disclosure provides for, and includes a medicament comprising a filtered extract of Candida albicans and secreted antigens formulated for reducing the diameter of a plurality of common warts by at least 50% at a cumulative dose of 1 unit of potency.

In an aspect, the present disclosure provides for, and includes a medicament comprising a filtered extract of Candida albicans and secreted antigens formulated for complete resolution of a non-common wart at a cumulative dose of 5 units of potency.

In an aspect, the present disclosure provides for, and includes a medicament comprising a filtered extract of Candida albicans and secreted antigens formulated for complete resolution of a previously treated common wart at a cumulative dose of 5 units of potency.

In an aspect, the present disclosure provides for, and includes a medicament comprising a filtered extract of Candida albicans and secreted antigens formulated for delaying the reappearance of the common wart upon resolution at a cumulative dose of 2.5 units of potency.

In an aspect, the present disclosure provides for, and includes, a method for reducing the level of IL-23 in a subject in need thereof, the method comprises administering one or more intralesional injections to the subject of an amount of a pharmaceutical composition at a cumulative dose of 1 unit of potency, where the pharmaceutical composition comprises a filtered extract of Candida albicans and secreted antigens. In an aspect, a subject is diagnosed with at least one common wart. In one aspect, a method of the present disclosure reduces the level of IL-23 for at least about 15% in a subject upon receipt of a cumulative dose of a pharmaceutical composition of the present disclosure when compared to a level of IL-23 measured in the subject before the administering step. In an aspect, a method of the present disclosure reduces the level of IL-23 for at least about 35% in a subject upon receipt of a cumulative dose of a pharmaceutical composition of the present disclosure when compared to a level of IL-23 measured in the subject before the administering step.

In an aspect, the present disclosure provides for, and includes, a method for completely resolving a common wart in a subject in need thereof, the method comprises reducing the level of IL-23 by at least about 15% in a subject in need thereof. In an aspect, the present disclosure provides for, and includes, a method for completely resolving a common wart in a subject in need thereof, the method comprises reducing the level of IL-23 by at least about 35% in a subject in need thereof. In one aspect, a method of the present disclosure reduces the level of IL-23 by administering one or more intralesional injections to the subject of an amount of a pharmaceutical composition at a cumulative dose of 3 units of potency, where the pharmaceutical composition comprises a filtered extract of Candida albicans and secreted antigens.

In an aspect, the present disclosure provides for, and includes, a method for reducing the level of IL-7 in a subject in need thereof, the method comprises administering one or more intralesional injections to the subject of an amount of a pharmaceutical composition at a cumulative dose of 0.6 units of potency, where the pharmaceutical composition comprises a filtered extract of Candida albicans and secreted antigens. In an aspect, a subject is diagnosed with at least one common wart. In one aspect, a method of the present disclosure reduces the level of IL-7 for at least about 10% in a subject upon receipt of a cumulative dose of a pharmaceutical composition of the present disclosure when compared to a level of IL-7 measured in the subject before the administering step.

In an aspect, the present disclosure provides for, and includes, a method for reducing the level of IL-7 in a subject in need thereof, the method comprises administering one or more intralesional injections to the subject of an amount of a pharmaceutical composition at a cumulative dose of 3 units of potency, where the pharmaceutical composition comprises a filtered extract of Candida albicans and secreted antigens. In an aspect, a subject is diagnosed with at least one common wart. In one aspect, a method of the present disclosure reduces the level of IL-7 for at least about 20% in the subject upon receipt of a cumulative dose of a pharmaceutical composition of the present disclosure when compared to a level of IL-7 measured in the subject before the administering step.

In an aspect, the present disclosure provides for, and includes, a method for reducing the level of IP-10 in a subject in need thereof, the method comprises administering one or more intralesional injections to the subject of an amount of a pharmaceutical composition at a cumulative dose of 3 units of potency, where the pharmaceutical composition comprises a filtered extract of Candida albicans and secreted antigens. In an aspect, a subject is diagnosed with at least one common wart. In one aspect, a method of the present disclosure reduces the level of IP-10 for at least about 5% in the subject upon receipt of a cumulative dose of a pharmaceutical composition of the present disclosure when compared to a level of IP-10 measured in the subject before the administering step.

In an aspect, the present disclosure provides for, and includes a medicament comprising a filtered extract of Candida albicans and secreted antigens used for reducing the level of IL-23 in a subject in need thereof. In an aspect, a subject is diagnosed with at least one common wart.

In an aspect, the present disclosure provides for, and includes a medicament comprising a filtered extract of Candida albicans and secreted antigens used for completely resolving a common wart in a subject in need thereof by reducing the level of IL-23 by at least about 35% in a subject in need thereof. In an aspect, the present disclosure provides for, and includes a medicament comprising a filtered extract of Candida albicans and secreted antigens used for completely resolving a common wart in a subject in need thereof by reducing the level of IL-23 by at least about 15% in a subject in need thereof.

In an aspect, the present disclosure provides for, and includes a medicament comprising a filtered extract of Candida albicans and secreted antigens used for reducing the level of IL-7 in a subject in need thereof. In an aspect, a subject is diagnosed with at least one common wart.

In an aspect, the present disclosure provides for, and includes a medicament comprising a filtered extract of Candida albicans and secreted antigens used for reducing the level of IP-10 in a subject in need thereof. In an aspect, a subject is diagnosed with at least one common wart.

### BRIEF DESCRIPTION OF THE DRAWINGS

The present disclosure is disclosed with reference to the accompanying drawings, where:

FIG. 1 shows characteristic morphology of isolated colonies of a first strain of Candida albicans, deposited as ATCC Accession No. PTA-126019, in accordance with the present disclosure;

FIG. 2 shows characteristic morphology of isolated colonies of a second strain of Candida albicans, deposited as ATCC Accession No. PTA-126020, in accordance with the present disclosure;

FIG. 3 shows typical appearance of budding yeast cells of Candida albicans in accordance with the present disclosure;

FIG. 4 shows typical appearance of pseudohyphae of Candida albicans in accordance with the present disclosure;

FIG. 5 illustrates an example of a track grid on the back of a guinea pig in accordance with the present disclosure; and

FIG. 6 illustrates the preferred location of the intralesional injection.

FIGS. 7A, 7B, and 7C illustrate comparison of changes in biomarker levels of IL-7, IP-10, and IL-23, respectively, within a treatment group and a placebo group in accordance with the present disclosure.

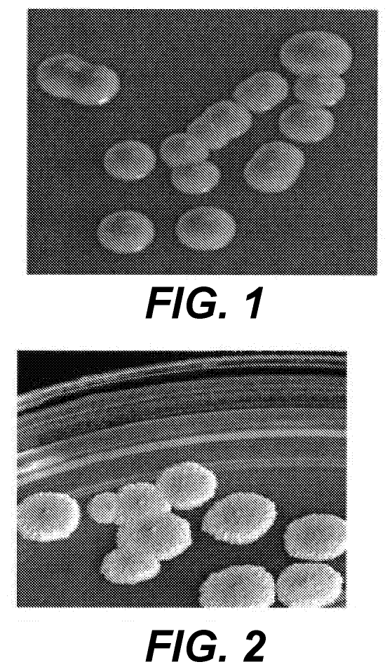

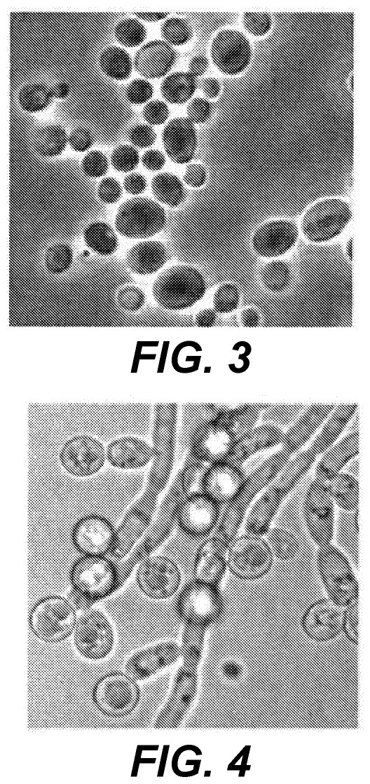

### DETAILED DESCRIPTION

The present disclosure provides for, and includes, pharmaceutical compositions having standardized potencies for use in the treatment of one or more common warts or non-common warts. In one aspect, treatment of one or more common warts or non-common warts refers to therapeutic treatment of these warts. In an aspect, treatment of one or more common warts or non-common warts refers to providing prophylactic or preventative measures against the development of warts. In an aspect, a pharmaceutical composition of the present disclosure is capable of complete resolution of one or more common warts and non-common warts with a cumulative dose given in units of potency in accordance with the present disclosure. In one aspect, a pharmaceutical composition of the present disclosure is capable of partial resolution of one or more common warts with a cumulative dose given in units of potency in accordance with the present disclosure. In an aspect, a pharmaceutical composition of the present disclosure is capable of delaying the reappearance of one or more common warts upon resolution with a cumulative dose given in units of potency in accordance with the present disclosure.

The present disclosure also provides for, and includes, methods for treating one or more common warts or non-common warts using the pharmaceutical compositions described herein. In an aspect, methods for completely resolving one or more common warts or non-common warts are provided. In one aspect, methods for partially resolving one or more common warts are provided. In an aspect, methods for treating previously treated common warts are provided. In one aspect, methods for delaying reappearance of common warts are provided.

Unless defined otherwise herein, terms are to be understood according to conventional usage by those of ordinary skill in the relevant art. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

All publications, patents, and patent applications mentioned in this disclosure are herein incorporated by reference to the same extent as if each individual publication, patent, or patent application was specifically and individually indicated to be incorporated by reference.

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination, or A, B, and C in combination.

As used herein, terms in the singular and the singular forms “a,” “an,” and “the,” for example, include plural referents unless the content clearly dictates otherwise.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure. Whenever the phrase “comprising” is used, variations such as “consisting essentially of” and “consisting of” are also contemplated.

The term “about,” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

As used herein, a “pharmaceutical composition” of the present disclosure can be an antigen or solvates thereof, described herein.

As used herein, an “excipient” refers to a substance formulated alongside the active ingredient of a medication. An excipient can be used for the purpose of long-term stabilization or to confer a therapeutic enhancement on the active ingredient in the final dosage form, such as facilitating active ingredient absorption, reducing viscosity, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life.

As used herein, “pharmaceutically acceptable carrier” includes any and all solvents, dispersion media, antibacterial and antifungal agents, isotonic and absorption delaying agents and the like. The use of such media and agents for pharmaceutically active substances is well known in the art. Except insofar as any conventional media or agent is incompatible with the pharmaceutical compositions, its use in pharmaceutical compositions is contemplated. As used herein, the phrase “pharmaceutically, pharmacologically, or physiologically acceptable” refers to molecular entities and compositions that do not produce adverse, allergic, or other untoward reactions when administered to an animal or a human. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof.

As used herein, the term “pharmaceutically acceptable solvate” refers to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. For example, a method or composition provided here can be used to delay the progression of a condition (e.g., warts). As used herein, “delaying” the progression of a condition means to defer, hinder, slow, retard, stabilize, and/or postpone development of the condition. This delay can be of varying lengths of time, depending on the history of the condition and/or individual being treated.

As used herein, the term “wart” refers to a growth on the skin caused by human papillomavirus.

As used herein, the term “anatomical region” refers to any one of the following regions of a subject's body: left arm, right arm, left hand, right hand, left leg, right leg, left foot, right foot, and torso. As used herein, an arm region includes a shoulder, upper and lower arms, a wrist, and half of an armpit. As used herein, a hand region starts from the distal to the wrist, excluding any periungual warts. As used herein, a foot starts from the distal to ankles, excluding the sole of the foot. As used herein, a leg region includes the upper leg, the lower leg, and an ankle. As used herein, a torso region includes the neck, the back, the chest, the abdomen, the hips, the buttocks and the pelvic region.

As used herein, the term “recalcitrant wart” refers to a wart that was not successfully treated by prior treatment, excluding over-the-counter treatments. As used herein, the term “over-the-counter treatment” refers to medications sold directly to a consumer without a prescription from a healthcare professional.

As used herein, the term “perimeter” refers to the peripheral margin of a wart.

As used herein, the term “recurrence” refers to the growth of a wart at the same location where a previously regressed wart was present.

As used herein, the term “complete resolution” refers to a sustained resolution of a wart reflected by a lack of recurrence of a wart as observed at least 20 weeks from administration of the first intralesional injection.

As used herein, the term “partial resolution” refers to any reduction in the diameter of a wart as compared to the diameter observed before the first intralesional injection.

As used herein, the terms “administer,” “administering,” or “administration” in reference to a dosage form of the disclosure refers to the act of introducing the dosage form into the system of subject in need of treatment. When a dosage form of the disclosure is given in combination with one or more other active agents (in their respective dosage forms), “administration” and its variants are each understood to include concurrent and/or sequential introduction of the dosage form and the other active agents. Administration of any of the described dosage forms includes simultaneous (parallel) administration, sequential (stepwise) administration, co-administration, or separate administration, in which the therapies are administered separately at approximately the same time, e.g., within about a few seconds to a few hours of one another.

As used herein, the term “medicament” refers to a substance used to treat an illness.

As used herein, the term “molecular weight” refers to the average mass of a molecule of interest to one-twelfth of the mass of carbon 12. A molecular weight is given in the unit of Daltons (Da). A molecular weight is measured by a method using a superpose 12 column calibrated with dextran standards having molecular weights between 1 kilodalton and 512 kilodalton.

As used herein, the term “IL-23” refers to interleukin 23, which is a heterodimeric cytokine having a p19 and p 40 subunit.

As used herein, the term “IL-7” refers to interleukin 7, which is a cytokine important for B and T cell development.

As used herein, the term “IP-10” refers to interferon gamma-induced protein, which is also known as CXCL10.

### A. Manufacturing Process of Pharmaceutical Compositions

In one aspect, a pharmaceutical composition of the present disclosure comprising a filtered extract of Candida albicans and secreted antigens can be prepared by a series of steps comprising growing two or more strains of Candida albicans separately, pooling cultures of two or more strains of Candida albicans and dialyzing the mixture, heating the mixture, lyophilizing the heated dialyzed material, producing a dry powder, extracting the dry powder, filtering the extract, and producing a master lot filtered solution. In an aspect, a pharmaceutical composition of the present disclosure can be produced by the protocol as outlined in Example 1.

In an aspect, a pharmaceutical composition of the present disclosure may be prepared using two or more strains of Candida albicans, such as using two strains, three strains, four strains, five strains, six strains, seven strains, eight strains, nine strains, or ten strains. In one aspect, a pharmaceutical composition of the present disclosure is prepared using two strains of Candida albicans. In an aspect, a pharmaceutical composition of the present disclosure is prepared using strains of Candida albicans provided in Hasenclever HF and Mitchell WO, “Antigenic Studies of Candida,” Journal of Bacteriology 1961; 82:578-581. In an aspect, a pharmaceutical composition of the present disclosure is prepared using two strains of Candida albicans, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In one aspect, a master lot filtered solution can be diluted at about 2:998 by volume with a diluent to form a pharmaceutical composition, meaning that 2 ml of a master lot filtered solution is diluted with 988 ml of a diluent. In an aspect, a master lot filtered solution can be diluted at a range of from about 2:998 to about 1:1 by volume with a diluent, such as from about 2:998 to about 10:990, from about 10:990 to about 20:980, from about 20:980 to about 30:970, from about 30:970 to about 40:960, from about 40:960 to about 50:950, from about 50:950 to about 60:940, from about 60:940 to about 70:930, from about 70:930 to about 80:920, from about 80:920 to about 90:910, from about 90:910 to about 1:9, from about 1:9 to about 1:8, from about 1:8 to about 1:7, from about 1:7 to about 1:6, from about 1:6 to about 1:5, from about 1:5 to about 1:4, from about 1:4 to about 1:3, from about 1:3 to about 1:2, from about 1:2 to about 1:1. In an aspect, a master lot filtered solution is undiluted in a pharmaceutical composition.

In an aspect, the present disclosure provides for a resulting pharmaceutical composition comprising at least 80% mannose, such as from 80% mannose to 85% mannose, from 85% mannose to 90% or mannose from 80% to 90% mannose. In an aspect, the present disclosure provides for a resulting pharmaceutical composition comprising at least 8% glucose, such as from 8% glucose to 10% glucose, from 10% glucose to 12% glucose, or from 8% glucose to 12% glucose. In an aspect, the present disclosure provides for a resulting pharmaceutical composition comprising at least 1% galactose, such as from 1% galactose to 2.5% galactose, from 2.5% galactose to 5% galactose, or from 1% to 5% galactose.

In an aspect, the present disclosure provides for a resulting pharmaceutical composition comprising antigens having a molecular weight of about 167 kilodaltons, such as from 157 kilodaltons to 177 kilodaltons. In an aspect, a molecular weight is measured by a method using a superpose 12 column calibrated with dextran standards having molecular weights between 1 kilodalton and 512 kilodalton.

### B. Test for Relative Potency

A unit of potency in accordance with the present disclosure is defined by a relative potency (RP) compared to a reference standard Candida albicans extract. A compound determined to have a relative potency of 1 compared to the standard is assigned to have a potency of 1 unit per mL. Relative potency is determined in a female IAF Hairless guinea pig (Crl:HA-HO) model, compared to a standard Candida albicans extract that is capable of eliciting an induration response >5 mm in an immunologically competent person at 48 hours after a 0.1 mL injection. In an aspect, a reference standard is established by skin test titration in humans with delayed-type hypersensitivity to Candida albicans.

Female IAF Hairless guinea pigs are selected to be as uniform in age and weight as possible. In an aspect, twenty or less female IAF Hairless guinea pigs can be used in a study. They are generally 4-6 months of age, and their body weights at treatment commencement will range from approximately 0.6-0.8 kilograms. All study animals are acclimatized to their designated housing at least 14 days prior to the first day of use.

At week 0, all study animals are sensitized with a 1:1 mixture of Complete Freund's Adjuvant (CFA) mixed with an antigen solution. In an aspect, an antigen solution is a 10-fold dilution of the standard Candida albicans extract using unpreserved saline. In another aspect, an antigen solution can be a 100-fold dilution of the standard Candida albicans extract using unpreserved saline. In an aspect, Adjulite CFA (Pacific Immunology) or any other commercially available CFA can be used in this assay. Animals are injected using a needle with a maximum of 4 subcutaneous injections of 0.1 ml, or 0.4 ml total, of the prepared mixture. In an aspect, a needle can be a 21 g needle, a 22 g needle, or a 23 g needle. These injections may be given in the cervical region (either the nape or over the shoulders), the gluteal (maximum of two injections in each location, one on each side), and/or the inguinal areas (a maximum of 0.1 ml may be given in each inguinal area). Any one injection regimen containing CFA can be applied to cach guinea pig in the study.

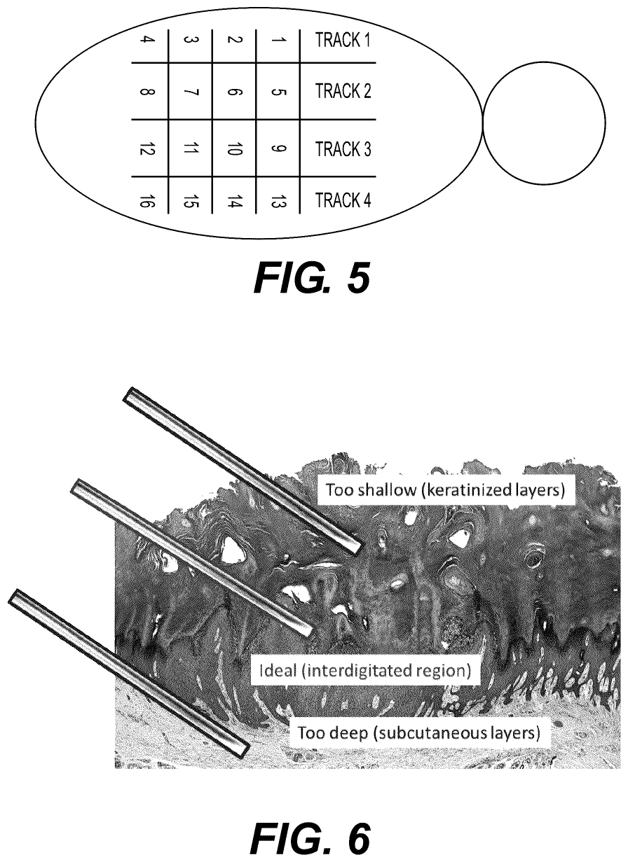

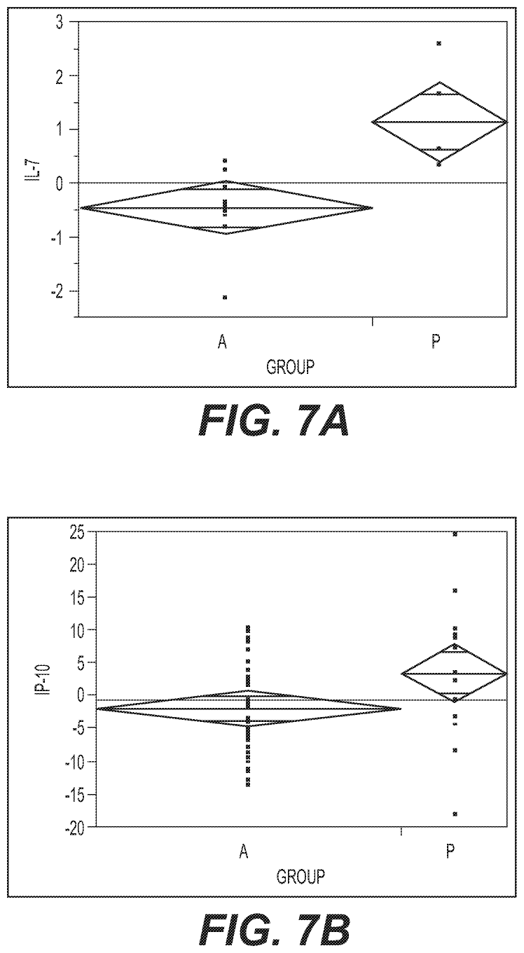

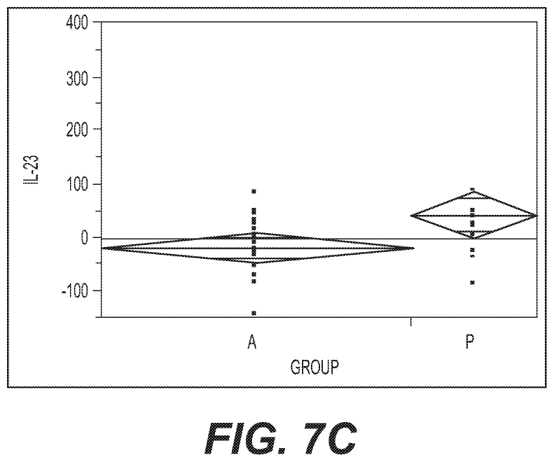

At week 4±1 week, four serial dilutions of each test sample and the standard are prepared independently with phenolated normal saline. For example, a set of serial dilutions can be prepared at 1:1, 1:4, 1:16, and 1:64. Animals are sedated for intradermal injection to ensure proper placement. Injections are given at a volume of 0.1 mL using a 27 g intradermal bevel needle on the backs of the sensitized guinea pigs. Injection locations are separated by at least 3 cm apart on a grid on the back of the guinea pigs. For example, four “tracks” can be outlined on the back of a guinea pig to for injections of a standard (Track 1) and three test samples (Tracks 2-4). Each of the “tracks” can be arranged such that the dilutions increase from the last diluted (positioned cranially), to the most diluted (positioned caudally), with all “tracks” being parallel to the spine. See FIG. 5 for an illustration of an example of a track grid on the back of a guinea pig.

At 24 hours±4 hours following injections, animals are restrained by hand, and indurations are marked and measured using a modified Mantoux procedure: With a ruler, the longest possible diameter (in mm) and its midpoint orthogonal diameter (in mm) of the indurated area are recorded. The reaction (response) at each test site is determined by averaging the longest and orthogonal axis measurements. Alternatively, a calibrated caliper may be used. Optionally, the above measurements can be repeated at 48 hours ±4 hours.

In an aspect, for each guinea pig, responses of Tracks 2, 3 and 4 vs Track 1 are plotted against their corresponding dilution and analyzed using the parallel line method: First, Track 2 and Track 1 response data against log(dilution) are plotted on the same graph, and the linearity of the responses in each track's data is tested, followed by the test for parallelism of the two lines. If the two lines are parallel, then RP2 is calculated using the equation Log(RP2)=(α₂−α₁)/β, where α₂ is the Y-intercept of Track 2, α₁ is the Y-intercept of Track 1, and β is the common slope of the plotted lines. The same calculation is repeated for Tracks 3 and 4 vs Track 1 to obtain RP3 and RP4, respectively, using the same equation above with α₂ replaced with either α₃ or α₄.

In an aspect, for each guinea pig, responses of Tracks 2, 3 and 4 vs Track 1 are plotted against their corresponding dilution and analyzed using the slope-ratio method: First, Track 2 and Track 1 response data against dilution are plotted on the same graph, the linearity of the responses in each track's data is tested with the regression model E(Y)=α+β D, where α denotes a common intercept Y-intercept shared by the two plots, βₜ denotes the slope of the test line (e.g. Track 2), and βᵣ denotes the slope of the reference line (e.g. Track 1). Two validity criteria must be met before using the slop-ratio method of analysis: (1) the slopes of the two tracks are significantly different from zero at a-level of 0.05; and (2) the intercept of the test line and that of the reference line are statistically equal, which means that the 90% confidence interval for the difference in the intercepts contains 0. If the above criteria are met, an estimate of relative potency, RP2, is calculated as RP2=estimate of (βₜ)/estimate of (βᵣ). The same calculation is repeated for Tracks 3 and 4 vs Track 1 to obtain RP3 and RP4, respectively, using the same equation above with α₂ replaced with either α₃ or α₄.

After RP2, RP3, and RP4 are obtained, these are plotted against the RP of Track 1. A regression analysis is performed to obtain the coefficient of determination denoted by R². The R² is targeted to be ≥0.85. Each of RP2, RP3, and RP4 are the relative potencies of the respective sample in that track.

A relative potency of 1 denotes that a test sample has the same potency as the reference standard.

### C. Formulation

One or more components of a pharmaceutical composition of the present disclosure can be formulated in admixture with conventional excipients, carriers, buffers, etc. In an aspect, an excipient is a substance formulated alongside the active ingredient of a medication. Typical carriers include, but are not limited to: water; salt solutions; alcohols; gum arabic; vegetable oils; benzyl alcohols; polyethylene glycols; gelatin; carbohydrates, such as lactose, amylose or starch; magnesium stearate; talc; silicic acid; paraffin; perfume oil; fatty acid esters; hydroxymethylcellulose; polyvinyl pyrrolidone; etc. In an aspect, a carrier includes any and all solvents, dispersion media, antibacterial and antifungal agents, isotonic and absorption delaying agents and the like.

One or more components of a pharmaceutical composition of the present disclosure can be sterilized and, if desired, mixed with auxiliary agents such as: lubricants; preservatives; disintegrants; stabilizers such as cyclodextrans; wetting agents; emulsifiers; salts; buffers; natural or artificial coloring agents; natural or artificial flavoring agents; or aromatic substances.

One or more components of a pharmaceutical composition of the present disclosure can also include one or more of the following agents: acetylated monoglyceride, aspartame, beta carotene, calcium stearate, carnauba wax, cellulose acetate phthalate, citric acid, citric acid anhydrous, colloidal silicon dioxide, confectioner's sugar, crospovidone, docusate sodium, cthyl alcohol, ferric oxide, fructose, gelatin, glycerine, glyceryl monostearate (e.g. glyceryl monostearate 40-50), glyceryl triacetate, HPMC (hydroxypropyl methylcellulose), hydroxypropyl cellulose, hypromellose, iron oxide, isopropyl alcohol, lactose monohydrate, low substituted hydroxypropyl cellulose, magnesium carbonate, magnesium stearate, maltol, mannitol, methacrylic acid, methacrylic acid copolymer (e.g. methacrylic acid copolymer type C), methylcellulose, microcrystalline cellulose, mono ammonium glycyrrhizinate, n-butyl alcohol, paraffin, pectin propylene glycol alginate, polyacrylate, polyethylene glycol (e.g. polyethylene glycol 6000), polysorbate 80, polyvinyl pyrrolidone, povidone, propylene glycol, shellac, silicon dioxide, sodium carbonate, sodium citrate, sodium hydroxide, sodium lauryl sulfate, sodium stearyl fumarate, sorbitol, starch, sucrose, sugar sphere, talc, titanium dioxide, tricthyl citrate, and xanthan gum.

In an aspect, a pharmaceutical composition described herein comprises an active compound prepared for administration as solutions of free base or pharmacologically acceptable salts in water suitably mixed with a surfactant, such as hydroxypropylcellulose. Pharmaceutically acceptable base addition salts can be formed with metals or amines, such as alkali and alkaline carth metals or organic amines. Pharmaceutically acceptable salts of compounds can also be prepared with a pharmaceutically acceptable cation. Suitable pharmaceutically acceptable cations are well known to those skilled in the art and include alkaline, alkaline carth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′dibenzylethylenediamine, chloroprocaine, choline, diethanolamine, dicyclohexylamine, ethylenediamine, N methylglucamine, and procaine.

Pharmaceutically acceptable acid addition salts include inorganic or organic acid salts. Examples of suitable acid salts include the hydrochlorides, acetates, citrates, salicylates, nitrates, phosphates. Other suitable pharmaceutically acceptable salts are well known to those skilled in the art and include, for example, acetic, citric, oxalic, tartaric, or mandelic acids, hydrochloric acid, hydrobromic acid, sulfuric acid or phosphoric acid; with organic carboxylic, sulfonic, sulfo or phospho acids or N substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4 aminosalicylic acid, 2 phenoxybenzoic acid, 2 acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2 hydroxyethanesulfonic acid, ethane 1,2 disulfonic acid, benzenesulfonic acid, 4 methylbenzenesulfoc acid, naphthalene 2 sulfonic acid, naphthalene 1,5 disulfonic acid, 2 or 3 phosphoglycerate, glucose 6 phosphate, N-cyclohexylsulfamic acid (with the formation of cyclamates), or with other acid organic compounds, such as ascorbic acid.

In an aspect, a pharmaceutical composition of the present disclosure comprises NaCl, NaHCO₃, human albumin, polysorbate 80, and phenol. In an aspect, a pharmaceutical composition of the present disclosure comprises 0.5% NaCl, 0.25% NaHCO₃, 0.03% human albumin, 8 ppm polysorbate 80, and 0.4% phenol.

In an aspect, a pharmaceutical composition of the present disclosure as described herein can be administered to the subject in need thereof via any route including, but not limited to, sublingual, caudal, dental, endocervical, enteral, epidural, extracorporeal, intravenous, implantation, infiltration, intra-amniotic, intra-arterial, intra-articular, intrabuccal, intra-cardiac, intra-caudal, intra-cavitary, intra-dermal, intra-discal, intralesional, intralymphatic, intraocular, intraperitoneal, intrapleural, intraspinal, intrasynovial, intrathecal, intratracheal, intratumor, intratympanic, intrauterine, intravascular, intravitreal, iontophoresis, irrigation, nasal, parenteral, percutaneous, periarticular, peridural, periodontal, photophoresis, retrobulbar, subarachnoid, subconjunctival, submucosal, transdermal, ureteral, and urethral. In one aspect, a pharmaceutical composition of the present disclosure is administered to the subject in need thereof intralesionally. In one aspect, administering is performed by the act of introducing the dosage form into the system of subject in need of treatment.

In an aspect, a pharmaceutical composition of the present disclosure as described herein can be administered via a form selected from the group consisting of oral, buccal, sublingual, topical, injectable, infused, inhalable, rectal, intravenous, intramuscular, and subcutaneous forms. For example, a pharmaceutical composition of the present disclosure as described herein can be administered as a transdermal patch, in a microneedle, as a mist, as suppository, as a gel, as a cream, as a pill, or as a spray using methods known in the art. In one aspect, a pharmaceutical composition of the present disclosure as described herein is administered via an injectable form.

A pharmaceutical composition provided here can be prepared as pharmaceutical forms suitable for injectable use. In an aspect, such compositions include sterile aqueous solutions or dispersions and sterile powders for the extemporaneous preparation of sterile injectable solutions or dispersions. In all cases the form is sterile and must be fluid to the extent that easy syringability exists. It remains stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. In an aspect, a solvent is a pharmaceutically acceptable solvate. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In an aspect, sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In an aspect, a pharmaceutical composition disclosed herein comprises human serum albumin. In one aspect, a pharmaceutical composition disclosed herein comprises polysorbate 80.

In an aspect, a pharmaceutical composition disclosed herein can be formulated as a sterile stabilized filtered extract of Candida albicans. In one aspect, a pharmaceutical composition can be formulated in a single dose vial. In an aspect, a pharmaceutical composition can be formulated in a single unpreserved vial. In one aspect, a pharmaceutical composition can be formulated in a multi-dose vial. In an aspect, a pharmaceutical composition can be formulated in a multi-dose preserved vial. In an aspect, a pharmaceutical composition can be formulated as a prefilled syringe or device with or without an attached needle. In an aspect, a pharmaceutical composition can be formulated in a container selected from the group consisting of a needle, a vial, and an ampoule. In an aspect, a container can be a glass container or a plastic container. In one aspect, a container has a volume of about 0.25 ml, about 0.5 ml, about 0.75 ml, about 1.0 ml, about 1.5 ml, about 2.0 ml, about 2.5 ml, about 3.0 ml, about 3.5 ml, about 4.0 ml, about 4.5 ml, about 5 ml, about 6 ml, about 7 ml, about 8 ml, about 9 ml, or about 10 ml.

In an aspect, a medicament comprising a filtered extract of Candida albicans and secreted antigens is provided for use in the treatment of warts according to the present disclosure at a cumulative dose provided by the present disclosure. In one aspect, a treatment of warts is selected from the group consisting of complete resolution of a common wart, partial resolution of a common wart, reducing the diameter of a common wart, partial resolution of a plurality of common warts, reducing the diameter of a plurality of common warts by at least 50%, complete resolution of a non-common wart, complete resolution of a previously treated common wart, and delaying the reappearance of the common wart upon resolution. In an aspect, a medicament provides a treatment of warts at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency. In an aspect, a medicament is formulated for administration to a subject by injection. In one aspect, an injection is provided as an intralesional injection.

In an aspect, a medicament comprising a filtered extract of Candida albicans and secreted antigens formulated for complete resolution of a common wart at a cumulative dose of 2.5 units of potency is provided. In one aspect, the present disclosure provides for a medicament comprising a filtered extract of Candida albicans and secreted antigens formulated for complete resolution of a common wart at a cumulative dose of less than about 0.5 unit of potency, less than about 1 unit of potency, less than about 2 units of potency, less than about 3 units of potency, less than about 4 units of potency, less than about 5 units of potency, less than about 6 unit of potency, less than about 7 units of potency, less than about 8 units of potency, less than about 9 units of potency, or less than about 10 units of potency.

In one aspect, a medicament comprising a filtered extract of Candida albicans and secreted antigens formulated for partial resolution of a common wart at a cumulative dose of 5 units of potency is provided. In one aspect, the present disclosure provides for a medicament comprising a filtered extract of Candida albicans and secreted antigens formulated for partial resolution of a common wart at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In an aspect, a medicament comprising a filtered extract of Candida albicans and secreted antigens formulated for reducing the diameter of a common wart by at least 50% at a cumulative dose of 1 unit of potency is provided. In one aspect, the present disclosure provides for a medicament comprising a filtered extract of Candida albicans and secreted antigens formulated for reducing the diameter of a common wart by at least 50% at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In one aspect, a medicament comprising a filtered extract of Candida albicans and secreted antigens formulated for partial resolution of a plurality of common warts at a cumulative dose of 5 units of potency is provided. In one aspect, the present disclosure provides for a medicament comprising a filtered extract of Candida albicans and secreted antigens formulated for partial resolution of a plurality of common warts at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In an aspect, a medicament comprising a filtered extract of Candida albicans and secreted antigens formulated for reducing the diameter of a plurality of common warts by at least 50% at a cumulative dose of 1 unit of potency is provided. In one aspect, the present disclosure provides for a medicament comprising a filtered extract of Candida albicans and secreted antigens formulated for reducing the diameter of a plurality of common warts by at least 50% at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In one aspect, a medicament comprising a filtered extract of Candida albicans and secreted antigens formulated for complete resolution of a non-common wart at a cumulative dose of 5 units of potency is provided. In one aspect, the present disclosure provides for a medicament comprising a filtered extract of Candida albicans and secreted antigens formulated for complete resolution of a non-common wart at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In an aspect, a medicament comprising a filtered extract of Candida albicans and secreted antigens formulated for complete resolution of a previously treated common wart at a cumulative dose of 5 units of potency is provided. In one aspect, the present disclosure provides for a medicament comprising a filtered extract of Candida albicans and secreted antigens formulated for complete resolution of a previously treated common wart at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In one aspect, a medicament comprising a filtered extract of Candida albicans and secreted antigens formulated for delaying the reappearance of the common wart upon resolution at a cumulative dose of 2.5 units of potency is provided. In one aspect, the present disclosure provides for a medicament comprising a filtered extract of Candida albicans and secreted antigens formulated for delaying the reappearance of the common wart upon resolution at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In an aspect, a medicament comprising a filtered extract of Candida albicans and secreted antigens used for reducing the level of a cytokine biomarker in a subject diagnosed with a common wart is provided. In one aspect, a cytokine biomarker is an inflammatory cytokine. In an aspect, a cytokine biomarker is selected from the group consisting of IL-23, IL-7, and IP-10. In an aspect, the level of a cytokine biomarker in a subject is reduced upon administration of a medicament comprising a filtered extract of Candida albicans and secreted antigens at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency. In an aspect, the level of a cytokine biomarker in a subject is reduced upon administration of a medicament comprising a filtered extract of Candida albicans and secreted antigens at a cumulative dose of at least about 0.5 unit of potency, at least about 1 unit of potency, at least about 2 units of potency, at least about 3 units of potency, at least about 4 units of potency, at least about 5 units of potency, at least about 6 units of potency, at least about 7 units of potency, at least about 8 units of potency, at least about 9 units of potency, or at least about 10 units of potency. In an aspect, the level of a cytokine biomarker in a subject is reduced upon administration of a medicament comprising a filtered extract of Candida albicans and secreted antigens at a cumulative dose from about 0.5 unit of potency to about 10 units of potency, such as from about 0.5 unit of potency to about 9.5 units of potency, from about 1 unit of potency to about 9.5 units of potency, from about 1.5 units of potency to about 9 units of potency, from about 2 units of potency to about 9 units of potency, from about 2 units of potency to about 8.5 units of potency, from about 2.5 units of potency to about 8.5 units of potency, from about 2.5 units of potency to about 8 units of potency, from about 3 units of potency to about 8 units of potency, from about 3 units of potency to about 7.5 units of potency, from about 3.5 units of potency to about 7.5 units of potency, from about 3.5 units of potency to about 7 units of potency, from about 4 units of potency to about 7 units of potency, from about 4 units of potency to about 6.5 units of potency, from about 4.5 units of potency to about 6.5 units of potency, from about 4.5 units of potency to about 6 units of potency, or from about 5 units of potency to about 6 units of potency. In an aspect, the level of a cytokine biomarker level is reduced up to 99.9% in a subject upon administration of a cumulative dose of a medicament, such as up to 99.5%, up to 99%, up to 98%, up to 95%, up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, up to 10%, or up to 5%. In one aspect, the level of a cytokine biomarker level is reduced from about 5% to about 99.9% in a subject upon administration of a cumulative dose of a medicament, such as from about 5% to about 99.8%, from about 5% to about 99.5%, from about 5% to about 99%, from about 5% to about 98%, from about 5% to about 95%, from about 10% to about 95%, from about 10% to about 90%, from about 15% to about 90%, from about 15% to about 85%, from about 20% to about 85%, from about 20% to about 80%, from about 25% to about 80%, from about 25% to about 75%, from about 30% to about 75%, from about 30% to about 70%, from about 35% to about 70%, from about 35% to about 65%, from about 40% to about 65%, from about 40% to about 60%, from about 45% to about 60%, from about 45% to about 55%, or from about 50% to about 55%.

In an aspect, a medicament comprising a filtered extract of Candida albicans and secreted antigens used for reducing the level of IL-23 in a subject in need thereof is provided. In an aspect, a subject is diagnosed with at least one common wart.

In an aspect, a medicament comprising a filtered extract of Candida albicans and secreted antigens used for completely resolving a common wart in a subject in need thereof by reducing the level of IL-23 by at least about 35% in a subject in need thereof is provided. In an aspect, a medicament comprising a filtered extract of Candida albicans and secreted antigens used for completely resolving a common wart in a subject in need thereof by reducing the level of IL-23 by at least about 15% in a subject in need thereof is provided.

In an aspect, a medicament comprising a filtered extract of Candida albicans and secreted antigens used for reducing the level of IL-7 in a subject in need thereof is provided. In an aspect, a subject is diagnosed with at least one common wart.

In an aspect, a medicament comprising a filtered extract of Candida albicans and secreted antigens used for reducing the level of IP-10 in a subject in need thereof is provided. In an aspect, a subject is diagnosed with at least one common wart.

In an aspect, a medicament for use in the complete resolution of a common wart is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in the partial resolution of a common wart is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in reducing the diameter of a common wart by at least 50% is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in the partial resolution of a plurality of common warts is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in reducing the diameter of a plurality of common warts by at least 50% is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in the complete resolution of a non-common wart is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in the complete resolution of a previously treated common wart is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament for use in delaying the reappearance of the common wart is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament comprising a filtered extract of Candida albicans and secreted antigens used for reducing the level of IL-23 in a subject in need thereof is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a subject is diagnosed with at least one common wart. In an aspect, a representative sample of a first strain has been deposited with the ATCC under ATCC Accession No. ATCC-10231.

In an aspect, a medicament comprising a filtered extract of Candida albicans and secreted antigens used for completely resolving a common wart in a subject in need thereof by reducing the level of IL-23 by at least about 35% in a subject in need thereof is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a medicament comprising a filtered extract of Candida albicans and secreted antigens used for reducing the level of IL-7 in a subject in need thereof is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a subject is diagnosed with at least one common wart. In an aspect, a representative sample of a first strain has been deposited with the ATCC under ATCC Accession No. ATCC-10231.

In an aspect, a medicament comprising a filtered extract of Candida albicans and secreted antigens used for reducing the level of IP-10 in a subject in need thereof is provided, where the medicament comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a subject is diagnosed with at least one common wart. In an aspect, a representative sample of a first strain has been deposited with the ATCC under ATCC Accession No. ATCC-10231.

In an aspect, a medicament comprising a filtered extract of Candida albicans and secreted antigens is formulated to have a potency of at least about 1 unit per ml, such as at least about 2 units per ml, at least about 3 units per ml, at least about 4 units per ml, at least about 5 units per ml, at least about 6 units per ml, at least about 7 units per ml, at least about 8 units per ml, at least about 9 units per ml, at least about 10 units per ml, at least about 15 units per ml, at least about 20 units per ml, at least about 25 units per ml, at least about 30 units per ml, at least about 40 units per ml, at least about 50 units per ml, at least about 30 units per ml, at least about 40 units per ml, at least about 50 units per ml, at least about 60 units per ml, at least about 70 units per ml, at least about 80 units per ml, at least about 90 units per ml, or at least about 100 units per ml.

### D. Method of Treatment

In an aspect, a method for treating a wart in a subject in need thereof is provided, the method comprises administering one or more doses of a pharmaceutical composition of the present disclosure to a subject in need thereof. In one aspect, a growth on the skin caused by human papillomavirus. In an aspect, a pharmaceutical composition comprises a filtered extract of Candida albicans and secreted antigens. In an aspect, a method of the present disclosure completely or partially arrests wart development. In one aspect, a method of the present disclosure completely or partially relieves a wart. In an aspect, a method of the present disclosure causes regression of a wart. In an aspect, a method of the present disclosure completely or partially prevents warts from developing in a patient that may be predisposed to wart development. In one aspect, a treatment of warts is selected from the group consisting of complete resolution of a common wart, partial resolution of a common wart, reducing the diameter of a common wart, partial resolution of a plurality of common warts, reducing the diameter of a plurality of common warts by at least 50%, complete resolution of a non-common wart, complete resolution of a previously treated common wart, and delaying the reappearance of the common wart upon resolution. In an aspect, a method for treating a wart achieves its treatment goal at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency. In an aspect, each dose is administered to a subject by injection. In one aspect, an injection is provided as an intralesional injection.

In an aspect, a method for treating a common wart in a subject in need thereof is provided, the method comprises administering one or more intralesional injections to the subject of an amount of a pharmaceutical composition effective for complete resolution of the common wart at a cumulative dose of 2.5 units of potency, where the pharmaceutical composition comprises a filtered extract of Candida albicans and secreted antigens. In one aspect, a method for treating a common wart in a subject in need thereof is provided, the method comprises administering one or more intralesional injections to the subject of an amount of a pharmaceutical composition capable of complete resolution of the common wart at a cumulative dose of 2.5 units of potency, where the pharmaceutical composition comprises a filtered extract of Candida albicans and secreted antigens.

In an aspect, complete resolution of a common wart is identified by a lack of recurrence of the common wart at the same site observed at least 20 weeks from administration of the first intralesional injection, such as observed at about 20 weeks, at about 25 weeks, at about 30 weeks, at about 35 weeks, at about 40 weeks, about 45 weeks, about 50 weeks, about 55 weeks, about 60 weeks, about 70 wecks, about 80 wecks, about 90 weeks, or about 100 weeks after administration of the first intralesional injection. In an aspect, recurrence of a common wart is defined by the growth of a wart at the same location where a previously regressed wart was present.

In an aspect, complete resolution of the wart is accompanied by a lack of scarring at the location of the common wart. In one aspect, complete resolution of the wart is accompanied by a low level of hypopigmentation at the location of the common wart, such as less than 3% of all resolved common warts.

In an aspect, a method for treating a common wart in a subject in need thereof is provided, the method comprises administering one or more intralesional injections to the subject of an amount of a pharmaceutical composition effective for partial resolution of the common wart at a cumulative dose of 5 units of potency, where the pharmaceutical composition comprises a filtered extract of Candida albicans and secreted antigens. In one aspect, a pharmaceutical composition effective for partial resolution of the common wart at a cumulative dose of 5 units of potency is also effective for reducing the diameter of the common wart by at least 50% at a cumulative dose of 1 unit of potency. In an aspect, partial resolution of a wart is identified by any reduction in the diameter of a wart as compared to the diameter observed before the first intralesional injection.

In an aspect, a method for treating a common wart in a subject in need thereof is provided, the method comprises administering one or more intralesional injections to the subject of an amount of a pharmaceutical composition capable of partial resolution of the common wart at a cumulative dose of 5 units of potency, where the pharmaceutical composition comprises a filtered extract of Candida albicans and secreted antigens. In one aspect, a pharmaceutical composition effective for partial resolution of the common wart at a cumulative dose of 5 units of potency is also capable of reducing the diameter of the common wart by at least 50% at a cumulative dose of 1 unit of potency.

In an aspect, a method for treating a plurality of common warts in a subject in need thereof is provided, the method comprises administering one or more intralesional injections to the subject of an amount of a pharmaceutical composition effective for partial resolution of the plurality of common warts at a cumulative dose of 5 units of potency, where the pharmaceutical composition comprises a filtered extract of Candida albicans and secreted antigens. In one aspect, a method for treating a plurality of common warts in a subject in need thereof is provided, the method comprises administering one or more intralesional injections to the subject of an amount of a pharmaceutical composition capable of partial resolution of the plurality of common warts at a cumulative dose of 5 units of potency, where the pharmaceutical composition comprises a filtered extract of Candida albicans and secreted antigens.

In an aspect, a method for treating a plurality of common warts in a subject in need thereof is provided, the method comprises administering one or more intralesional injections to the subject of an amount of a pharmaceutical composition effective for reducing the diameter of each of the plurality of common warts by at least 50% at a cumulative dose of 1 unit of potency, where the pharmaceutical composition comprises a filtered extract of Candida albicans and secreted antigens. In an aspect, a method for treating a plurality of common warts in a subject in need thereof is provided, the method comprises administering one or more intralesional injections to the subject of an amount of a pharmaceutical composition capable of reducing the diameter of each of the plurality of common warts by at least 50% at a cumulative dose of 1 unit of potency, where the pharmaceutical composition comprises a filtered extract of Candida albicans and secreted antigens.

In one aspect, a plurality of common warts comprise 2 to 20 common warts, such as 2 to 20 common warts, 2 to 19 common warts, 3 to 19 common warts, 3 to 18 common warts, 4 to 18 common warts, 4 to 17 common warts, 5 to 17 common warts, 5 to 16 common warts, 6 to 16 common warts, 6 to 15 common warts, 7 to 15 common warts, 7 to 14 common warts, 8 to 14 common warts, 8 to 13 common warts, 9 to 13 common warts, 9 to 12 common warts, 10 to 12 common warts, 10 to 11 common warts, or 11 to 12 common warts. In an aspect, a plurality of common warts are located within the same anatomical location in a subject. In one aspect, a plurality of common warts are located in different anatomical locations in a subject. In an aspect, an anatomical location is selected from one of the following regions of a subject's body: left arm, right arm, left hand, right hand, left leg, right leg, left foot, right foot, and torso. In one aspect, an arm region includes a shoulder, upper and lower arms, a wrist, and half of an armpit. In an aspect, a hand region starts from the distal to the wrist, excluding any periungual warts. In one aspect, a foot starts from the distal to ankles, excluding the sole of the foot. In an aspect, a leg region includes the upper leg, the lower leg, and an ankle. In one aspect, a torso region includes the neck, the back, the chest, the abdomen, the hips, the buttocks and the pelvic region.

In an aspect, a method for treating a non-common wart in a subject in need thereof is provided, where the subject has one or more common warts, and the method comprises administering one or more intralesional injections to the subject of an amount of a pharmaceutical composition effective for complete resolution of the non-common wart at a cumulative dose of 5 units of potency, where the pharmaceutical composition comprises a filtered extract of Candida albicans and secreted antigens. In an aspect, a method for treating a non-common wart in a subject in need thereof is provided, where the subject has one or more common warts, and the method comprises administering one or more intralesional injections to the subject of an amount of a pharmaceutical composition capable of complete resolution of the non-common wart at a cumulative dose of 5 units of potency, where the pharmaceutical composition comprises a filtered extract of Candida albicans and secreted antigens.

In an aspect, a non-common wart is a plantar wart. In one aspect, a non-common wart is a genital wart. In an aspect, a non-common wart is a facial wart. In one aspect, a non-common wart is a flat wart. In one aspect, a non-common wart is a periungual wart. In an aspect, a non-common wart is located within the same anatomical area as one or more common warts.

In an aspect, complete resolution of a non-common wart is identified by a lack of recurrence of the non-common wart at the same site observed at least 20 weeks from administration of the first intralesional injection, such as observed at about 20 weeks, at about 25 wecks, at about 30 weeks, at about 35 weeks, at about 40 weeks, about 45 weeks, about 50 weeks, about 55 weeks, about 60 weeks, about 70 weeks, about 80 weeks, about 90 weeks, or about 100 weeks after administration of the first intralesional injection. In an aspect, recurrence of a common wart is defined by the growth of a wart at the same location where a previously regressed wart was present.

In one aspect, a method for treating a previously treated common wart in a subject in need thereof is provided, the method comprises administering one or more intralesional injections to the subject of an amount of a pharmaceutical composition effective for complete resolution of the previously treated common wart at a cumulative dose of 5 units of potency, where the pharmaceutical composition comprises a filtered extract of Candida albicans and secreted antigens. In an aspect, a method for treating a previously treated common wart in a subject in need thereof is provided, the method comprises administering one or more intralesional injections to the subject of an amount of a pharmaceutical composition capable of complete resolution of the previously treated common wart at a cumulative dose of 5 units of potency, where the pharmaceutical composition comprises a filtered extract of Candida albicans and secreted antigens.

In an aspect, a previously treated common wart is a common wart previously treated with cryotherapy. In one aspect, a previously treated common wart did not respond to cryotherapy. In an aspect, a cryotherapy did not completely resolve a previously treated common wart.

In one aspect, a previously treated common wart is a common wart previously treated with salicylic acid or a related acid. In an aspect, a related acid is trichloroacetic acid or bichloroacetic acid. In an aspect, a previously treated common wart did not respond to salicylic acid or a related acid. In one aspect, salicylic acid or a related acid did not completely resolve a previously treated common wart.

In an aspect, a previously treated common wart is a common wart previously treated with a treatment selected from the group consisting of liquid nitrogen, carbon dioxide, cantharidin, simple occlusion, wart gel, apple cider vinegar, surgery, laser, tea tree oil, freeze wart spray, wart scraped, electrodessication, essential oils of lavender and oregano, and imiquimod. In one aspect, a previously treated common wart did not respond to a prior treatment. In an aspect, a prior treatment did not completely resolve a previously treated common wart.

In an aspect, complete resolution of a previously treated common wart is identified by a lack of recurrence of the previously treated common wart at the same site observed at least 20 weeks from administration of the first intralesional injection, such as observed at about 20 weeks, at about 25 weeks, at about 30 weeks, at about 35 weeks, at about 40 weeks, about 45 weeks, about 50 weeks, about 55 weeks, about 60 weeks, about 70 weeks, about 80 weeks, about 90 weeks, or about 100 weeks after administration of the first intralesional injection. In an aspect, recurrence of a common wart is defined by the growth of a wart at the same location where a previously regressed wart was present.

In an aspect, complete resolution of the previously treated common wart is accompanied by a lack of scarring at the location of the common wart. In one aspect, complete resolution of the previously treated common wart is accompanied by a low level of hypopigmentation at the location of the common wart, such as less than 3% of all resolved common warts.

In one aspect, a method for delaying recurrence of a common wart in a subject in need thereof is provided, the method comprises administering one or more intralesional injections to the subject of an amount of a pharmaceutical composition effective for delaying the reappearance of the common wart upon resolution at a cumulative dose of 2.5 units of potency, where the pharmaceutical composition comprises a filtered extract of Candida albicans and secreted antigens. In one aspect, a method for delaying recurrence of a common wart in a subject in need thereof is provided, the method comprises administering one or more intralesional injections to the subject of an amount of a pharmaceutical composition capable of delaying the reappearance of the common wart upon resolution at a cumulative dose of 2.5 units of potency, where the pharmaceutical composition comprises a filtered extract of Candida albicans and secreted antigens. In an aspect, delaying the recurrence of a common wart means to defer, hinder, slow, retard, stabilize, and/or postpone development of the condition at the same location where a previously regressed wart was present. In one aspect, the delay can be of varying lengths of time, depending on the history of the condition and/or individual being treated. In an aspect, a subject does not develop any new common warts within at least 16 weeks after the last injection of the treatment regimen, such as at least 18 weeks, at least 20 weeks, at least 25 weeks, at least 30 weeks, at least 35 weeks, at least 40 weeks, at least 45 weeks, at least 50 weeks, at least 55 weeks, at least 60 weeks, at least 65 weeks, at least 70 weeks, at least 80 weeks, at least 90 weeks, or at least 100 weeks after the last injection of the treatment regimen. In an aspect, a subject does not develop any new common warts within the same anatomical area of the resolved common wart. In one aspect, a subject does not develop any new common warts at the same site as the resolved common wart.

In an aspect, a method for reducing the level of a cytokine biomarker in a subject in need thereof, the method comprises administering one or more intralesional injections to the subject of an amount of a pharmaceutical composition at a cumulative dose of from about 0.5 units of potency to about 10 units of potency, where the pharmaceutical composition comprises a filtered extract of Candida albicans and secreted antigens. In one aspect, a cytokine biomarker is an inflammatory cytokine. In an aspect, a subject is diagnosed with at least one common wart. In an aspect, a cytokine biomarker is selected from the group consisting of IL-23, IL-7, and IP-10. In an aspect, the level of a cytokine biomarker in a subject is reduced upon administration of a pharmaceutical composition of the present disclosure at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency. In an aspect, the level of a cytokine biomarker in a subject is reduced upon administration of a pharmaceutical composition of the present disclosure at a cumulative dose of at least about 0.5 unit of potency, at least about 1 unit of potency, at least about 2 units of potency, at least about 3 units of potency, at least about 4 units of potency, at least about 5 units of potency, at least about 6 units of potency, at least about 7 units of potency, at least about 8 units of potency, at least about 9 units of potency, or at least about 10 units of potency. In an aspect, the level of a cytokine biomarker in a subject is reduced upon administration of a pharmaceutical composition of the present disclosure at a cumulative dose from about 0.5 unit of potency to about 10 units of potency, such as from about 0.5 unit of potency to about 9.5 units of potency, from about 1 unit of potency to about 9.5 units of potency, from about 1.5 units of potency to about 9 units of potency, from about 2 units of potency to about 9 units of potency, from about 2 units of potency to about 8.5 units of potency, from about 2.5 units of potency to about 8.5 units of potency, from about 2.5 units of potency to about 8 units of potency, from about 3 units of potency to about 8 units of potency, from about 3 units of potency to about 7.5 units of potency, from about 3.5 units of potency to about 7.5 units of potency, from about 3.5 units of potency to about 7 units of potency, from about 4 units of potency to about 7 units of potency, from about 4 units of potency to about 6.5 units of potency, from about 4.5 units of potency to about 6.5 units of potency, from about 4.5 units of potency to about 6 units of potency, or from about 5 units of potency to about 6 units of potency. In an aspect, the level of a cytokine biomarker level is reduced up to 99.9% in a subject upon administration of a cumulative dose of a pharmaceutical composition of the present disclosure, such as up to 99.5%, up to 99%, up to 98%, up to 95%, up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, up to 10%, or up to 5%. In one aspect, the level of a cytokine biomarker level is reduced from about 5% to about 99.9% in a subject upon administration of a cumulative dose of a pharmaceutical composition of the present disclosure, such as from about 5% to about 99.8%, from about 5% to about 99.5%, from about 5% to about 99%, from about 5% to about 98%, from about 5% to about 95%, from about 10% to about 95%, from about 10% to about 90%, from about 15% to about 90%, from about 15% to about 85%, from about 20% to about 85%, from about 20% to about 80%, from about 25% to about 80%, from about 25% to about 75%, from about 30% to about 75%, from about 30% to about 70%, from about 35% to about 70%, from about 35% to about 65%, from about 40% to about 65%, from about 40% to about 60%, from about 45% to about 60%, from about 45% to about 55%, or from about 50% to about 55%.

In an aspect, a method for reducing the level of IL-23 in a subject I need thereof is provided, the method comprises administering one or more intralesional injections to the subject of an amount of a pharmaceutical composition at a cumulative dose of 1 unit of potency, where the pharmaceutical composition comprises a filtered extract of Candida albicans and secreted antigens. In an aspect, a subject is diagnosed with at least one common wart. In one aspect, a method of the present disclosure reduces the level of IL-23 for at least about 15% in a subject upon receipt of a cumulative dose of a pharmaceutical composition of the present disclosure when compared to a level of IL-23 measured in the subject before the administering step. In one aspect, a method of the present disclosure reduces the level of IL-23 for at least about 35% in a subject upon receipt of a cumulative dose of a pharmaceutical composition of the present disclosure when compared to a level of IL-23 measured in the subject before the administering step.

In an aspect, a method for completely resolving a common wart in a subject in need thereof is provided, the method comprises reducing the level of IL-23 by at least about 35% in a subject in need thereof. In an aspect, a method for completely resolving a common wart in a subject in need thereof is provided, the method comprises reducing the level of IL-23 by at least about 15% in a subject in need thereof. In one aspect, a method of the present disclosure reduces the level of IL-23 by administering one or more intralesional injections to the subject of an amount of a pharmaceutical composition at a cumulative dose of 3 units of potency, where the pharmaceutical composition comprises a filtered extract of Candida albicans and secreted antigens.

In an aspect, a method for reducing the level of IL-7 in a subject in need thereof is provided, the method comprises administering one or more intralesional injections to the subject of an amount of a pharmaceutical composition at a cumulative dose of 0.6 units of potency, where the pharmaceutical composition comprises a filtered extract of Candida albicans and secreted antigens. In an aspect, a subject is diagnosed with at least one common wart. In one aspect, a method of the present disclosure reduces the level of IL-7 for at least about 10% in a subject upon receipt of a cumulative dose of a pharmaceutical composition of the present disclosure when compared to a level of IL-7 measured in the subject before the administering step.

In an aspect, a method for reducing the level of IL-7 in a subject in need thereof is provided, the method comprises administering one or more intralesional injections to the subject of an amount of a pharmaceutical composition at a cumulative dose of 3 units of potency, where the pharmaceutical composition comprises a filtered extract of Candida albicans and secreted antigens. In an aspect, a subject is diagnosed with at least one common wart. In one aspect, a method of the present disclosure reduces the level of IL-7 for at least about 20% in the subject upon receipt of a cumulative dose of a pharmaceutical composition of the present disclosure when compared to a level of IL-7 measured in the subject before the administering step.

In an aspect, a method for reducing the level of IP-10 in a subject in need thereof is provided, the method comprises administering one or more intralesional injections to the subject of an amount of a pharmaceutical composition at a cumulative dose of 3 units of potency, where the pharmaceutical composition comprises a filtered extract of Candida albicans and secreted antigens. In an aspect, a subject is diagnosed with at least one common wart. In one aspect, a method of the present disclosure reduces the level of IP-10 for at least about 5% in the subject upon receipt of a cumulative dose of a pharmaceutical composition of the present disclosure when compared to a level of IP-10 measured in the subject before the administering step.

In an aspect, methods of the present disclosure are effective at achieving a treatment goal at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency. In one aspect, methods of the present disclosure are capable of achieving a treatment goal at a cumulative dose of less than about 0.5 unit of potency, less than about 1 unit of potency, less than about 2 units of potency, less than about 3 units of potency, less than about 4 units of potency, less than about 5 units of potency, less than about 6 units of potency, less than about 7 units of potency, less than about 8 units of potency, less than about 9 units of potency, or less than about 10 units of potency.

In an aspect, methods of the present disclosure are effective at achieving a treatment goal at a cumulative dose of at least about 0.5 unit of potency, at least about 1 unit of potency, at least about 2 units of potency, at least about 3 units of potency, at least about 4 units of potency, at least about 5 units of potency, at least about 6 units of potency, at least about 7 units of potency, at least about 8 units of potency, at least about 9 units of potency, or at least about 10 units of potency. In one aspect, methods of the present disclosure are capable of achieving a treatment goal at a cumulative dose of at least about 0.5 unit of potency, at least about 1 unit of potency, at least about 2 units of potency, at least about 3 units of potency, at least about 4 units of potency, at least about 5 units of potency, at least about 6 units of potency, at least about 7 units of potency, at least about 8 units of potency, at least about 9 units of potency, or at least about 10 units of potency.

In an aspect, methods of the present disclosure are effective at achieving a treatment goal at a cumulative dose from about 0.5 unit of potency to about 10 units of potency, such as from about 0.5 unit of potency to about 9.5 units of potency, from about 1 unit of potency to about 9.5 units of potency, from about 1.5 units of potency to about 9 units of potency, from about 2 units of potency to about 9 units of potency, from about 2 units of potency to about 8.5 units of potency, from about 2.5 units of potency to about 8.5 units of potency, from about 2.5 units of potency to about 8 units of potency, from about 3 units of potency to about 8 units of potency, from about 3 units of potency to about 7.5 units of potency, from about 3.5 units of potency to about 7.5 units of potency, from about 3.5 units of potency to about 7 units of potency, from about 4 units of potency to about 7 units of potency, from about 4 units of potency to about 6.5 units of potency, from about 4.5 units of potency to about 6.5 units of potency, from about 4.5 units of potency to about 6 units of potency, or from about 5 units of potency to about 6 units of potency. In an aspect, methods of the present disclosure are capable of achieving aa treatment goal at a cumulative dose from about 0.5 unit of potency to about 10 units of potency, such as from about 0.5 unit of potency to about 9.5 units of potency, from about 1 unit of potency to about 9.5 units of potency, from about 1.5 units of potency to about 9 units of potency, from about 2 units of potency to about 9 units of potency, from about 2 units of potency to about 8.5 units of potency, from about 2.5 units of potency to about 8.5 units of potency, from about 2.5 units of potency to about 8 units of potency, from about 3 units of potency to about 8 units of potency, from about 3 units of potency to about 7.5 units of potency, from about 3.5 units of potency to about 7.5 units of potency, from about 3.5 units of potency to about 7 units of potency, from about 4 units of potency to about 7 units of potency, from about 4 units of potency to about 6.5 units of potency, from about 4.5 units of potency to about 6.5 units of potency, from about 4.5 units of potency to about 6 units of potency, or from about 5 units of potency to about 6 units of potency.

In an aspect, each dose administered in the methods of the present disclosure can be administered via a form selected from the group consisting of oral, buccal, sublingual, topical, injectable, infused, inhalable, rectal, intravenous, intramuscular, and subcutaneous forms. In an aspect, two or more intralesional injections are provided to a subject in need thereof over a period of time. In an aspect, cach dose has a potency of at least 0.3 unit of potency. In one aspect, cach dose has a potency of at least 0.5 unit of potency. In an aspect, each dose is administered at a dose of at least about 0.1 unit of potency to about 1 unit of potency, such as about 0.1 unit of potency to about 0.9 unit of potency, such as about 0.2 unit of potency to about 1 unit of potency, such as about 0.2 unit of potency to about 0.8 unit of potency, such as about 0.2 unit of potency to about 0.5 unit of potency, such as about 0.2 unit of potency to about 0.4 unit of potency, such as about 0.3 unit of potency to about 0.7 unit of potency, or such as about 0.4 unit of potency to about 0.6 unit of potency. In an aspect, each dose is provided at a volume of from about 5 μL to about 500 μL, such as from about 5 μL to about 450 μL, from about 5 μL to about 400 μL, from about 5 μL to about 350 μL, from about 5 μL to about 300 μL, from about 5 μL to about 250 μL, from about 5 μL to about 200 μL, from about 5 μL to about 150 μL, from about 5 μL to about 100 μL, from about 5 μL to about 50 μL, from about 5 μL to about 40 μL, from about 5 μL to about 30 μL, from about 5 μL to about 20 μL, from about 5 μL to about 10 μL, from about 50 μL to about 500 μL, from about 50 μL to about 450 μL, from about 50 μL to about 400 μL, from about 50 μL to about 350 μL, from about 50 μL to about 300 μL, from about 50 μL to about 250 μL, from about 50 μL to about 200 μL, from about 50 μL to about 150 μL, from about 50 μL to about 100 μL, from about 100 μL to about 500 μL, from about 150 μL to about 450 μL, from about 200 μL to about 400 μL, or from about 250 μL to about 350 μL. In one aspect, a pair of doses are provided about two weeks apart to a subject in need thereof, such as 10 days, 11 days, 12 days, 13 days, 14 days, 15 days, 16 days, 17 days, or 18 days apart. In an aspect, a pair of doses are provided about three weeks apart to a subject in need thereof, such as 17 days, 18 days, 19 days, 20 days, 21 days, 22 days, 23 days, 24 days, or 25 days apart. In an aspect, doses administered in the methods are provided to a subject in need thereof over a period of about 2 weeks to about 27 weeks, such as about 2 weeks to about 26 weeks, such as about 2 weeks to about 25 weeks, such as about 2 weeks to about 24 weeks, such as about 2 weeks to about 23 weeks, such as about 2 weeks to about 22 weeks, such as about 2 weeks to about 21 weeks, such as about 2 weeks to about 20 weeks, such as about 2 weeks to about 19 weeks, such as about 2 weeks to about 18 weeks, such as about 2 weeks to about 17 weeks, such as about 2 weeks to about 16 weeks, such as about 2 weeks to about 15 weeks, such as about 2 weeks to about 14 weeks, such as about 2 weeks to about 13 weeks, such as about 2 weeks to about 12 weeks, such as about 2 weeks to about 11 weeks, such as about 2 weeks to about 10 weeks, such as about 2 weeks to about 9 weeks, such as about 2 weeks to about 8 weeks, such as about 2 weeks to about 7 weeks, such as about 2 weeks to about 6 weeks, such as about 2 weeks to about 5 weeks, such as about 2 weeks to about 4 weeks, or such as about 2 weeks to about 3 weeks. In an aspect, doses administered in the methods are provided to a subject in need thereof over a period of about 3 weeks to about 27 weeks, such as about 4 weeks to about 27 weeks, about 5 weeks to about 27 weeks, about 6 weeks to about 27 weeks, about 7 weeks to about 27 weeks, about 8 weeks to about 27 weeks, about 9 weeks to about 27 weeks, about 10 weeks to about 27 weeks, about 11 weeks to about 27 weeks, about 12 weeks to about 27 weeks, about 13 weeks to about 27 weeks, about 14 weeks to about 27 weeks, about 15 weeks to about 27 weeks, about 16 weeks to about 27 weeks, about 17 weeks to about 27 weeks, about 18 weeks to about 27 weeks, about 19 weeks to about 27 weeks, about 20 weeks to about 27 weeks, about 21 weeks to about 27 weeks, about 22 weeks to about 27 weeks, about 23 weeks to about 27 weeks, about 24 weeks to about 27 weeks, about 25 weeks to about 27 weeks, or about 26 weeks to about 27 weeks. In one aspect, doses administered in the methods are provided to a subject in need thereof over a period of about 2 weeks, about 3 weeks, about 4 weeks, about 5 weeks, about 6 weeks, about 7 wecks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, or about 27 weeks.

In an aspect, an intralesional injection is provided to a subject in need thereof. In one aspect, an intralesional injection is provided at the perimeter of a common wart being treated. In an aspect, an intralesional injection is provided near the perimeter of a common wart being treated. In one aspect, an intralesional injection is provided at a location that is about 5 mm or less from the peripheral margin of a common wart being treated, such as about 4 mm or less, about 3 mm or less, about 2 mm or less, or about 1 mm or less from the peripheral margin of a common wart. In one aspect, a perimeter is defined by the peripheral margin of a wart. In an aspect, an intralesional injection is provided to the common wart being treated. In one aspect, an intralesional injection is provided to the largest common wart within an anatomical area, in the presence of a plurality of common warts in the same anatomical area. In one aspect, an intralesional injection is provided to the largest common wart within an anatomical area, in the presence of a plurality of common warts in the same anatomical area and a non-common wart. In an aspect, the largest common wart is determined by choosing the common wart within an anatomical area having the largest diameter.

In an aspect, a subject has a common wart measuring between about 3 mm to about 20 mm prior to beginning of a treatment of the present disclosure, such as between about 3 mm to about 19 mm, between about 4 mm to about 20 mm, between about 4 mm to about 19 mm, between about 5 mm to about 19 mm, between about 5 mm to about 18 mm, between about 6 mm to about 18 mm, between about 6 mm to about 17 mm, between about 7 mm to about 16 mm, between about 7 mm to about 15 mm, between about 8 mm to about 15 mm, between about 8 mm to about 14 mm, between about 9 mm to about 14 mm, between about 9 mm to about 13 mm, between about 10 mm to about 13 mm, between about 10 mm to about 12 mm, or between about 11 mm to about 12 mm. In an aspect, a subject has a common wart measuring between about 20 mm to about 1 cm, such as between about 20 mm to about 95 mm, such as between about 25 mm to about 1 cm, such as between about 20 mm to about 90 mm, such as between about 25 mm to about 95 mm, such as between about 25 mm to about 90 mm, such as between about 30 mm to about 90 mm, such as between about 30 cm to about 85 mm, such as between about 35 mm to about 85 mm, such as between about 35 mm to about 80 mm, such as between about 40 mm to about 80 mm, such as between about 40 mm to about 75 mm, such as between about 45 mm to about 75 mm, such as between about 45 mm to about 60 mm, such as between about 50 mm to about 70 mm, such as between about 50 mm to about 65 mm, such as between about 55 mm to about 65 mm, or such as between about 55 mm to about 60 mm. In an aspect, a subject has a common wart measuring between about 1 cm to about 100 cm, such as between about 1 cm to about 95 cm, between about 5 cm to about 100 cm, between about 5 cm to about 95 cm, between about 10 cm to about 95 cm, between about 10 cm to about 90 cm, between about 15 cm to about 90 cm, between about 15 cm to about 85 cm, between about 20 cm to about 85 cm, between about 20 cm to about 80 cm, between about 25 cm to about 80 cm, between about 25 cm to about 75 cm, between about 30 cm to about 75 cm, between about 30 cm to about 70 cm, between about 35 cm to about 70 cm, between about 35 cm to about 65 cm, between about 40 cm to about 65 cm, between about 40 cm to about 60 cm, between about 45 cm to about 60 cm, between about 45 cm to about 55 cm, or between about 50 cm to about 55 cm.

In an aspect, two or more intralesional injections are provided to a subject in need thereof over a period of time. In one aspect, each of the two or more intralesional injection is provided at a dose of at least 0.5 unit of potency. In an aspect, each of the two or more intralesional injection is provided at a dose of at least about 0.1 unit of potency to about 1 unit of potency, such as about 0.1 unit of potency to about 0.9 unit of potency, such as about 0.2 unit of potency to about 1 unit of potency, such as about 0.2 unit of potency to about 0.8 unit of potency, such as about 0.3 unit of potency to about 0.7 unit of potency, or such as about 0.4 unit of potency to about 0.6 unit of potency. In an aspect, each of the two or more intralesional injection is provided at a volume of from about 5 μL to about 500 μL, such as from about 5 μL to about 450 μL, from about 5 μL to about 400 μL, from about 5 μL to about 350 μL, from about 5 μL to about 300 μL, from about 5 μL to about 250 μL, from about 5 μL to about 200 μL, from about 5 μL to about 150 μL, from about 5 μL to about 100 μL, from about 5 μL to about 50 μL, from about 5 μL to about 40 μL, from about 5 μL to about 30 μL, from about 5 μL to about 20 μL, from about 5 μL to about 10 μL, from about 50 μL to about 500 μL, from about 50 μL to about 450 μL, from about 50 μL to about 400 μL, from about 50 μL to about 350 μL, from about 50 μL to about 300 μL, from about 50 μL to about 250 μL, from about 50 μL to about 200 μL, from about 50 μL to about 150 μL, from about 50 μL to about 100 μL, from about 100 μL to about 500 μL, from about 150 μL to about 450 μL, from about 200 μL to about 400 μL, or from about 250 μL to about 350 μL.

In one aspect, a pair of intralesional injections in two or more intralesional injections are provided about two weeks apart to a subject in need thereof, such as 10 days, 11 days, 12 days, 13 days, 14 days, 15 days, 16 days, 17 days, or 18 days apart. In an aspect, a pair of intralesional injections in two or more intralesional injections are provided about three weeks apart to a subject in need thereof, such as 17 days, 18 days, 19 days, 20 days, 21 days, 22 days, 23 days, 24 days, or 25 days apart.

In an aspect, two or more intralesional injections are provided to a subject in need thereof over a period of about 2 weeks to about 27 weeks, such as about 2 weeks to about 26 weeks, such as about 2 weeks to about 25 weeks, such as about 2 weeks to about 24 weeks, such as about 2 weeks to about 23 weeks, such as about 2 weeks to about 22 weeks, such as about 2 weeks to about 21 weeks, such as about 2 weeks to about 20 weeks, such as about 2 weeks to about 19 weeks, such as about 2 weeks to about 18 weeks, such as about 2 weeks to about 17 weeks, such as about 2 weeks to about 16 weeks, such as about 2 weeks to about 15 weeks, such as about 2 weeks to about 14 weeks, such as about 2 weeks to about 13 weeks, such as about 2 weeks to about 12 weeks, such as about 2 weeks to about 11 weeks, such as about 2 weeks to about 10 weeks, such as about 2 weeks to about 9 weeks, such as about 2 weeks to about 8 weeks, such as about 2 weeks to about 7 weeks, such as about 2 weeks to about 6 weeks, such as about 2 weeks to about 5 weeks, such as about 2 weeks to about 4 weeks, or such as about 2 weeks to about 3 weeks. In an aspect, two or more intralesional injections are provided to a subject in need thereof over a period of about 3 weeks to about 27 weeks, such as about 4 weeks to about 27 weeks, about 5 weeks to about 27 weeks, about 6 weeks to about 27 weeks, about 7 weeks to about 27 weeks, about 8 weeks to about 27 weeks, about 9 weeks to about 27 weeks, about 10 weeks to about 27 weeks, about 11 weeks to about 27 weeks, about 12 weeks to about 27 weeks, about 13 weeks to about 27 weeks, about 14 weeks to about 27 weeks, about 15 weeks to about 27 weeks, about 16 weeks to about 27 weeks, about 17 weeks to about 27 weeks, about 18 weeks to about 27 weeks, about 19 weeks to about 27 weeks, about 20 weeks to about 27 weeks, about 21 weeks to about 27 wecks, about 22 weeks to about 27 weeks, about 23 weeks to about 27 weeks, about 24 weeks to about 27 weeks, about 25 weeks to about 27 weeks, or about 26 weeks to about 27 weeks. In one aspect, two or more intralesional injections are provided to a subject in need thereof over a period of about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, or about 27 weeks.

In one aspect, two or more intralesional injections are provided to a subject in need thereof in two or more subgroups of intralesional injections over a period of time. In one aspect, cach of the subgroup of two or more subgroups is provided at a dose of at least 0.5 unit of potency. In an aspect, each of the subgroup of the two or more subgroups is provided at a dose of at least about 0.1 unit of potency to about 1 unit of potency, such as about 0.1 unit of potency to about 0.9 unit of potency, such as about 0.2 unit of potency to about 1 unit of potency, such as about 0.2 unit of potency to about 0.8 unit of potency, such as about 0.3 unit of potency to about 0.7 unit of potency, or such as about 0.4 unit of potency to about 0.6 unit of potency. In an aspect, cach of the subgroup of the two or more subgroups is provided at a volume of from about 5 μL to about 500 μL, such as from about 5 μL to about 450 μL, from about 5 μL to about 400 μL, from about 5 μL to about 350 μL, from about 5 μL to about 300 μL, from about 5 μL to about 250 μL, from about 5 μL to about 200 μL, from about 5 μL to about 150 μL, from about 5 μL to about 100 μL, from about 5 μL to about 50 μL, from about 5 μL to about 40 μL, from about 5 μL to about 30 μL, from about 5 μL to about 20 μL, from about 5 μL to about 10 μL, from about 50 μL to about 500 μL, from about 50 μL to about 450 μL, from about 50 μL to about 400 μL, from about 50 μL to about 350 μL, from about 50 μL to about 300 μL, from about 50 μL to about 250 μL, from about 50 μL to about 200 μL, from about 50 μL to about 150 μL, from about 50 μL to about 100 μL, from about 100 μL to about 500 μL, from about 150 μL to about 450 μL, from about 200 μL to about 400 μL, or from about 250 μL to about 350 μL.

In an aspect, a pair of subgroups within two or more subgroups of intralesional injections are provided about two weeks apart to a subject in need thereof, such as 10 days, 11 days, 12 days, 13 days, 14 days, 15 days, 16 days, 17 days, or 18 days apart. In one aspect, a pair of subgroups within two or more subgroups of intralesional injections are provided about three weeks apart to a subject in need thereof, such as 17 days, 18 days, 19 days, 20 days, 21 days, 22 days, 23 days, 24 days, or 25 days apart.

In an aspect, a subgroup of intralesional injections comprises at least two intralesional injections such as two intralesional injections, three intralesional injections, four intralesional injections, five intralesional injections, six intralesional injections, seven intralesional injections, eight intralesional injections, nine intralesional injections, or ten intralesional injections.

In one aspect, intralesional injections within a subgroup of intralesional injections are administered at approximately the same time, such as within about 1 minute, within about 2 minutes, within about 3 minutes, within about 4 minutes, within about 5 minutes. In one aspect, intralesional injections within a subgroup of intralesional injections are administered are administered within 24 hours, such as within about 18 hours, within about 12 hours, within about 6 hours, within about 5 hours, within about 4 hours, within about 3 hours, within about 2 hours, or within about 1 hour.

In one aspect, intralesional injections within a subgroup of intralesional injections are provided around a common wart being treated. In an aspect, intralesional injections within a subgroup of intralesional injections are evenly spaced apart near the perimeter of a common wart being treated. In one aspect, each of the intralesional injection within a subgroup of intralesional injections is provided at a location that is about 5 mm or less from the peripheral margin of a common wart being treated, such as about 4 mm or less, about 3 mm or less, about 2 mm or less, or about 1 mm or less from the peripheral margin of a common wart. In an aspect, intralesional injections within a subgroup of intralesional injections are evenly spaced apart at the perimeter of a common wart being treated. In one aspect, a perimeter is defined by the peripheral margin of a wart.

In an aspect, a pharmaceutical composition administered by a method of the present disclosure comprises a filtered extract of two strains of Candida albicans and secreted antigens, where a representative sample of a first strain has been deposited with the ATCC under ATCC Accession No. PTA-126019, and a representative sample of a second strain has been deposited with the ATCC under ATCC Accession No. PTA-126020. In an aspect, a representative sample of a first strain has been deposited with the ATCC under ATCC Accession No. ATCC-10231.

In an aspect, a subject being treated with the methods of the present disclosure is between the ages of 18 and 65, such as between 20 and 60, between 18 and 30, between 25 and 50, between 30 and 40, between 40 to 50, or between 50 to 65. In one aspect, a subject being treated with the methods of the present disclosure is a pediatric patient. In one aspect, a pediatric patient is a premature newborn. In an aspect, a pediatric patient is a term newborn. In one aspect, a pediatric patient is a neonate. In one aspect, a pediatric patient is an infant. In an aspect, a pediatric patient is a toddler. In one aspect, a pediatric patient is a young child. In one aspect, a pediatric patient is a child. In an aspect, a pediatric patient is an adolescent. In an aspect, a pediatric patient is a child patient below about 18, 15, 12, 10, 8, 6, 4, 3, 2, or 1 years old. In one aspect, a subject being treated with the methods of the present disclosure was diagnosed with a first common wart at least 12 weeks prior to receiving one or more intralesional injections, such as between 12 weeks to 100 weeks, between 15 weeks to 90 weeks, between 20 to 80 weeks, between 25 to 75 weeks, between 30 to 70 weeks, between 35 to 65 weeks, between 40 to 60 weeks, between 45 to 55 weeks, or between 50 to 55 weeks. In an aspect, a subject being treated with the methods of the present disclosure was not diagnosed with a recalcitrant wart. In an aspect, a recalcitrant wart is a wart that was not successfully treated by prior treatment, excluding over-the-counter treatments. In one aspect, a recalcitrant wart is a wart that was not successfully treated by one or more types of prior treatment, such as two or more, three or more, four or more, five or more, six or more, seven or more, eight or more, nine or more, or ten or more types of prior treatment. In an aspect, an over-the-counter treatment is a medication sold directly to a consumer without a prescription from a healthcare professional.

In an aspect, a subject being treated with the methods of the present disclosure has a baseline result of between 5 mm and 25 mm to the Delayed Type Hypersensitivity (DTH) test. In one aspect, a DTH test is performed by administering a single intradermal injection of CANDIN® (0.1 mL; Allermed/Nielsen Biosciences) on the volar surface of the forearm or on the outer aspect of the upper arm, at least 2 cm away from any primary injectable warts. The skin is cleansed with 70% alcohol before applying the skin test. The intradermal injection is given as superficially as possible causing a distinct, sharply defined bleb. The test is read at 48±4 hours post the DTH challenge injection, by visually inspecting the test site and palpating the indurated area. Measurements are made across two diameters. The mean of the longest and the midpoint orthogonal diameters of the indurated area is reported as the DTH response.

In an aspect, a subject being treated with the methods of the present disclosure is not diagnosed with a systematic disease that compromises immune function. In an aspect, a subject being treated with the methods of the present disclosure is not diagnosed with a localized disease that compromises immune function. In an aspect, a subject being treated with the methods of the present disclosure is not diagnosed with a systematic condition that compromises immune function. In an aspect, a subject being treated with the methods of the present disclosure is not diagnosed with a localized condition that compromises immune function. In an aspect, a subject being treated with the methods of the present disclosure is not diagnosed with psoriasis. In an aspect, a subject being treated with the methods of the present disclosure is not receiving a treatment resulting in being immunocompromised.

In an aspect, a subject being treated with the methods of the present disclosure has not been diagnosed with diabetes mellitus. In an aspect, a subject being treated with the methods of the present disclosure does not have a history of keloid formation. In an aspect, a subject being treated with the methods of the present disclosure does not have an existing dermatologic condition in the same anatomical area as the wart being treated. In an aspect, a subject being treated with the methods of the present disclosure does not have an underlying inflammatory condition. In an aspect, an underlying inflammatory condition is an arthritic joint.

In an aspect, a subject being treated with the methods of the present disclosure has not received one or more treatments selected from the group consisting of liquid nitrogen, carbon dioxide, electrodessication, laser, surgery, simple occlusion (e.g. duct tape), salicylic or related acids including trichloroacetic acid and bichloroacetic acid, over-the-counter treatments, and cantharidin, within 4 weeks prior to the administering step. In an aspect, a subject being treated with the methods of the present disclosure has not received one or more immunotherapy selected from the group consisting of diphenylcyclopropenone (DPCP), dinitrochlorobenzene (DNCB), imiquimod, 5-florouracil, bleomycin, and podophyllin, within 12 weeks prior to the administering step. In an aspect, a subject being treated with the methods of the present disclosure has not received one or more systematic treatment selected from the group consisting of cimetidine, zinc supplements at a dose higher than 20 mg of elemental zinc daily, azathioprine, 6-mercaptopurine, methotrexate, infliximab, adalimumab, etanercept, and steroid, within 12 weeks prior to the administering step. In an aspect, a subject being treated with the methods of the present disclosure has not received any investigational agent within 30 days prior to the administering step. In an aspect. a subject being treated with the methods of the present disclosure has not received any investigational agent within 5 half-lives of said investigational agent prior to the administering step.

1.-323. (canceled)

324. A method of treating an inflammatory disease, disorder, or condition in a subject in need thereof, the method comprising administering to the subject a pharmaceutical composition comprising a cumulative dose of between 0.5 to 10 units of potency of two or more strains of Candida albicans and secreted antigens and a pharmaceutically acceptable carrier or diluent, wherein each of the two or more strains is selected from the group consisting of a strain having been deposited with the ATCC under Accession No. PTA-126019, a strain having been deposited with the ATCC under Accession No. PTA-126020, and a strain having been deposited with the ATCC under Accession No. ATCC-10231.

325. The method of claim 324, wherein the pharmaceutical composition has a cumulative dose of between 0.5 to 5 units of potency.

326. The method of claim 324, wherein the pharmaceutical composition has a cumulative dose of between 0.5 to 2.5 units of potency.

327. The method of claim 324, wherein the pharmaceutical composition has a cumulative dose selected from the group consisting of about 10 units of potency, about 5 units of potency, about 2.5 units of potency, about 1 unit of potency, and about 0.5 unit of potency.

328. The method of claim 324, wherein the pharmaceutical composition comprises a first strain having been deposited under ATCC Accession No. PTA-126019, and a second strain having been deposited under ATCC Accession No. PTA-126020.

329. The method of claim 324, wherein the pharmaceutical composition comprises a first strain having been deposited under ATCC Accession No. ATCC-10231, and a second strain having been deposited under ATCC Accession No. PTA-126020.

330. The method of claim 324, wherein the pharmaceutical composition further comprises at least 80% mannose.

331. The method of claim 324, wherein the pharmaceutical composition further comprises at least 8% glucose.

332. The method of claim 324, wherein the pharmaceutical composition further comprises at least 1% galactose.

333. The method of claim 324, wherein the pharmaceutical composition is formulated as a sterile stabilized filtered extract.

334. The method of claim 324, wherein the pharmaceutically acceptable carrier or diluent comprises
i. 0.5% sodium chloride (NaCl),
ii. 0.25% sodium bicarbonate (NaHCO₃),
iii. 0.4% phenol,
iv. 0.03% human serum albumin, and
v. 8 parts per million (ppm) polysorbate 80.

335. The method of claim 324, wherein the administering is by injection.

336. The method of claim 324, wherein the administering is by topical administration, transdermal administration, or subcutaneous administration.

337. The method of claim 324, wherein the inflammatory disease, disorder, or condition is characterized by an elevated level of one or more inflammatory cytokines in the subject in need thereof.

338. The method of claim 337, wherein the elevated level of one or more inflammatory cytokines is reduced following administration of the pharmaceutical composition to the subject in need thereof.

339. The method of claim 338, wherein the one or more inflammatory cytokines are selected from the group consisting of interleukin 23 (IL-23), interleukin 7 (IL-7), and interferon gamma-induced protein (IP-10).

340. A transdermal patch comprising a pharmaceutical composition comprising a cumulative dose of between 0.5 to 10 units of potency comprising a sterile filtered extract of two or more strains of Candida albicans and secreted antigens and a pharmaceutically acceptable carrier or diluent, wherein each of the two or more strains of Candida albicans is selected from the group consisting of a strain having been deposited with the American Type Culture Collection (ATCC) under ATCC Accession No. PTA-126019, a strain having been deposited under ATCC Accession No. PTA-126020, and a strain having been deposited under ATCC Accession No. ATCC-10231, wherein the pharmaceutical composition is effective for partial or complete resolution of one or more warts, and wherein the transdermal patch is configured to deliver the pharmaceutical composition to a subject in need thereof.

341. The transdermal patch of claim 340, wherein the pharmaceutical comprises a first strain having been deposited under ATCC Accession No. PTA-126019, and a second strain having been deposited under ATCC Accession No. PTA-126020.

342. A microneedle comprising a pharmaceutical composition comprising a cumulative dose of between 0.5 to 10 units of potency comprising a sterile filtered extract of two or more strains of Candida albicans and secreted antigens and a pharmaceutically acceptable carrier or diluent, wherein each of the two or more strains of Candida albicans is selected from the group consisting of a strain having been deposited with the American Type Culture Collection (ATCC) under ATCC Accession No. PTA-126019, a strain having been deposited under ATCC Accession No. PTA-126020, and a strain having been deposited under ATCC Accession No. ATCC-10231, wherein the pharmaceutical composition is effective for partial or complete resolution of one or more warts, and wherein the microneedle is configured to deliver the pharmaceutical composition to a subject in need thereof.

343. The microneedle of claim 342, wherein the pharmaceutical comprises a first strain having been deposited under ATCC Accession No. PTA-126019, and a second strain having been deposited under ATCC Accession No. PTA-126020.

344. The microneedle of claim 342, wherein the microneedle is a plurality of microneedles.
